(19) United States
(12) Patent Application Publication
(10) Pub. No.: US 2003/0072758 A1
(43) Pub. Date: Apr. 17, 2003

(54) BMPR1A involvement in juvenile polyposis

(76) Inventor: James R. Howe, Iowa City, IA (US)

Correspondence Address: FULLBRIGHT & JAWORSKI L.L.P., 600 CONGRESS AVE., SUITE 2400, AUSTIN, TX, 78701, US

(21) Appl. No.: 10/153,217

(22) Filed: May 21, 2002

Related U.S. Application Data
(60) Provisional application No. 60/292,691, filed on May 21, 2001.

(51) Int. Cl. C12Q 1/68; G01N 33/574; A61K 39/395

(52) U.S. Cl. 424/155.1; 435/6; 435/7.23

(57) ABSTRACT

Familial juvenile polyposis is an autosomal dominant disease characterized by a predisposition to hamartomatous polyps and gastrointestinal cancer. The present invention shows that JP families carry germline mutations in BMPR1A, a gene located at 10q22-23. Methods and compositions for the detection and amelioration of FJP and gastrointestinal tumors are provided.

This application is related to, and claims a benefit of priority from, copending provisional U.S. Provisional Serial No. 60/292,691, filed May 21, 2001, the entire contents of which are hereby expressly incorporated by reference for all purposes.

### BACKGROUND OF THE INVENTION

1. Field of the Invention

The present invention relates to the fields of oncology, genetics and molecular biology. More particularly, the invention relates to the identification of the gene responsible for familial juvenile polyposis. Defects in this gene are associated with a predisposition to gastrointestinal cancers.

2. Description of Related Art

Colorectal cancer is the second leading cause of cancer death in the United States, and was responsible for 57,407 deaths in 1994 (Landis et al., 1998). Approximately 5-10% of the nearly 131,600 new colorectal cancer cases each year will involve a clear heritable predisposition, of which the majority involve hereditary non-polyposis colorectal cancer (HNPCC). About 1% of new colorectal cancers are related to inherited polyposis syndromes, which include familial adenomatous polyposis (FAP) and familial juvenile polyposis (FJP) (Rustgi, 1994).

Identification of the genes responsible for HNPCC and FAP have greatly increased the understanding of the molecular mechanisms contributing to the development of both familial and sporadic colorectal cancer. The intense study of sporadic colorectal carcinogenesis over the last decade has shown that these tumors develop through the multi-step accumulation of different genetic mutations within colonic epithelial cells (Vogelstein et al., 1988). Genes known to be involved in this progression include APC and MCC on 5q21, KRAS2 on 12p12, p53 at 17p13, and several mismatch repair genes as seen in HNPCC (reviewed in Howe and Guillem, 1997). Deletions on 18q21 also are quite common, occurring in approximately 75% of colorectal cancers (Vogelstein et al., 1988). Initial studies suggested that the tumor suppressor gene DCC (deleted in colorectal cancer)was the predisposing gene from this region (Fearon et al., 1990), but this has not been clearly established by further investigation.

Familial juvenile polyposis (JP) is an autosomal dominant condition characterized by multiple juvenile polyps of the gastrointestinal (GI) tract. Kindreds have been described in which there is involvement of the colon only (juvenile polyposis coli, MIM 174900) (Veale et al., 1966; Grotsky et al., 1982; Rozen and Baratz 1982), the upper GI tract (Watanabe et al., 1979), and both upper and lower GI tracts (generalized polyposis) (Sachatello et al., 1970; Stemper et al., 1975; Jarvinen and Franssila 1984), although whether these are distinct clinical entities is not clear. Affected family members often present with blood per rectum or anemia in the second decade of life (Jass et al., 1988).

Microscopically, the polyps contain cystically dilated glands, abundant stroma, and an inflammatory infiltrate (Morson 1962). There have been many reports of patients with juvenile polyposis developing gastrointestinal malignancy, including colon cancer (Stemper et al., 1975; Liu et al., 1978; Goodman et al., 1979; Rozen and Baratz 1982; Jarvinen and Franssila 1984; Ramaswamy et al., 1984; Baptist and Sabatini 1985; Jones et al., 1987; Bentley et al., 1989; Scott-Conner et al., 1995), stomach cancer (Stemper et al., 1975; Yoshida et al., 1988; Scott-Conner et al., 1995), and pancreatic cancer (Stemper et al., 1975; Walpole and Cullity, 1989). Affected family members' risk of developing GI malignancy has been estimated to be from 9% (Jarvinen and Franssila 1984) to as high as 50% (Jass, 1990). Development of adenocarcinoma has been hypothesized to begin with an adenomatous focus within a juvenile polyp, which later becomes dysplastic, and finally undergoes malignant transformation (Goodman et al., 1979; Jarvinen and Franssila, 1984).

JP is a hamartomatous polyposis syndrome, as are Peutz-Jegher's Syndrome (PJS) and Cowden's disease (CD). Although the polyps in PJS are true hamartomata, some may undergo adenomatous change, and these family members are at increased risk for gastrointestinal malignancy. The PJS gene was mapped to chromosome 19p by comparative genomic hybridization and linkage (Hemminki et al., 1997; Mehenni et al., 1997), and germline mutations were identified in the serine threonine kinase gene LKB1 (Hemminki et al., 1998). In CD, affected family members may develop multiple hamartomata of the skin, breast, thyroid, oral mucosa, or GI tract, and they are at risk for breast and thyroid malignancies. The gene for CD was localized to chromosome 10q22-23 by linkage (Nelen et al., 1996), and germline mutations in the PTEN gene have been found in affected family members (Liaw et al., 1997). A third entity, termed the “hereditary mixed-polyposis syndrome” (HMPS), differs from these syndromes in that affected family members have a typical juvenile polyps, colonic adenomas, and colorectal carcinomas. A gene for HMPS has been mapped to chromosome 6q by linkage (Thomas et al., 1996), and it remains uncertain whether HMPS is a distinct clinical syndrome or a variant of FJP (Whitelaw et al., 1997).

To date linkage studies in JP families have been limited, with one report excluding APC and MCC as the genes for FJP (Leggett et al., 1993). Other genetic studies, originally stimulated by the finding of an interstitial deletion at 10q22-24 in an infant with multiple colonic juvenile polyps and several congenital abnormalities (Jacoby et al., 1997b), have focused on the region of the PTEN gene. Evaluation for loss of heterozygosity in this region within juvenile polyps revealed somatic deletions within the lamina propria in 39 (83%) of 47 polyps derived from 13 unrelated patients with familial JP and 3 patients with sporadic juvenile polyps. These findings have been interpreted as evidence for a tumor-suppressor gene on 10q for FJP (termed “JP1”) (Jacoby et al., 1997a), but a recent study of fourteen FJP families found neither mutations in PTEN nor evidence of linkage to markers on 10q22-24 (Marsh et al., 1997). Analysis of an additional eleven cases of FJP also did not uncover mutations in the PTEN gene (Riggins et al., 1997). Lynch et al. (1977) reported one family thought to have both juvenile polyposis syndrome and CD as having a nonsense mutation in PTEN, and Olschwang et al. (1998) described three patients with juvenile polyposis as having PTEN mutations. Whether these four individuals should truly be considered as having juvenile polyposis rather than CD is not clear from these reports.

In 1998, the present inventor showed that a subset of juvenile polyposis families carry germ line mutations in the gene SMAD4 (also known as DPC4), located on chromosome 18q21.1. Howe et al. (1998b). This gene encodes a critical cytoplasmic mediator in the TGF-β signaling pathway. The mutant SMAD4 proteins were predicted to be truncated at the carboxyl-terminus and, hence, lacking sequences required for normal function. Howe et al (1998b). However, SMAD4 mutations only occur in about 20% of JP cases, leaving many other cases unassigned.

It is evident from the discussion presented above that FJP is a significant disease. The identification of additional genes involved in FJP will allow a more accurate determination of the molecular basis of gastrointestinal polyposis predisposing to colorectal cancer, as well as improved presymptomatic diagnosis of family members at risk. Such genes also may be involved in the genesis of sporadic colorectal cancers, and therefore their discovery could ultimately impact on the treatment of this large group of patients.

### SUMMARY OF THE INVENTION

Thus, in accordance with the present invention, there is provided method of diagnosing juvenile polyposis comprising the steps of (i) obtaining a sample from a subject; and (ii) determining the loss or alteration of a functional BMPR1A gene in cells of the sample. The sample may be tissue or fluid, such as blood, buccal smear or amniocentesis sample. Determining may comprise assaying for a nucleic acid from the sample, including amplifying the nucleic acid. Alternatively, determining may comprises contacting the sample with an antibody that binds immunologically to a BMPR1A, such as in an ELISA.

The method may further comprise comparing the expression of BMPR1A in the sample with the expression of BMPR1A in non-juvenile polyposis samples, for example, by evaluating the level of BMPR1A expression, or the structure of the BMPR1A gene, protein or transcript. Suitable assays for comparison include sequencing, wild-type oligonucleotide hybridization, mutant oligonucleotide hybridization, SSCP, PCR™, denaturing gradient gel electrophoresis and RNase protection. In particular, evaluating comprises wild-type or mutant oligonucleotide hybridization, for example, using an oligonucleotide array on a chip or wafer.

Specific mutations to be identified include a mutation in the coding sequence of BMPR1A, such as a deletion, an insertion, a frameshift, a nonsense mutation, a missense mutation or splice mutation. A particular type of frameshift mutation results in a premature termination of the BMPR1A gene product. Specific missense mutations include amino acid changes of C82Y, Q239X, W271X, Q117X, E84X, Y62D, and A338D. DNA changes include 245G→A, 715C→T, 812G→A, 349C→T, 262G→T, 184T→G, and 1013C→A. Premature stop mutations include those at residues 122-123, residues 363-364, residues 259-260, residues 35-36 and reisdue 321-322. DNA changes include 44-47delTGTT, 961delC, 353delT, and 1061delG. The mutations alos include loss of an exon splice site, for example, such as exon 7 splice caused by mutations 864-868delACTTG and IVS7+1−2delgt.

In an additional embodiment, the method comprising combining the BMPR1A diagnostic method with an subject is characterized by one or more of (a) five juvenile polyps of the colorectum; (b) junvile polyps throughout the gastrointestinal tract; and/or (c) a family history of juvenile polyposis. The subject may be characterized by two of (a)-(c), i.e., (a) and (b), (a) and (c) or (b) and (c). The subject also may be characterized by each of (a)-(c).

In another embodiment, there is provided a method for altering the phenotype of a cell in a subject having juvenile polyposis comprising the step of contacting the cell with a functional BMPR1A under conditions permitting the uptake of the BMPR1A by the cell. The cell may be derived from a gastrointestinal cell. The phenotype may be selected from the group consisting of proliferation, migration, contact inhibition, soft agar growth and cell cycling. The BMPR1A may be encapsulated in a liposome.

In yet another embodiment, there is provided a method for altering the phenotype of a cell in a subject having juvenile polyposis comprising the step of contacting the cell with a nucleic acid (i) encoding BMPR1 A and (ii) a promoter active in the cell, wherein the promoter is operably linked to the region encoding the BMPR1A, under conditions permitting the uptake of the nucleic acid by the cell. The cell may be derived from a gastrointestinal cell or a tumor cell. The phenotype may be proliferation, migration, contact inhibition, soft agar growth or cell cycling. The nucleic acid may be encapsulated in a liposome. The nucleic acid may be a viral vector selected from the group consisting of retrovirus, adenovirus, adeno-associated virus, vaccinia virus and herpesvirus, which may be encapsulated in a viral particle or a lipsome.

In still yet another embodiment, there is provided a method for treating juvenile polyposis comprising the step of contacting a cell within a subject with BMPR1A under conditions permitting the uptake of the BMPR1A by the cell. The subject may be a human.

In a further embodiment, there is provided a A method for treating juvenile polyposis in a subject comprising the step of contacting a cell within the subject with a nucleic acid (i) encoding BMPR1A and (ii) a promoter active in the cell, wherein the promoter is operably linked to the region encoding the BMPR1A, under conditions permitting the uptake of the nucleic acid by the cell. The cell may be derived from a tissue selected from the group consisting of skin, muscle, fascia, brain, prostate, breast, endometrium, lung, head & neck, pancreas, small intestine, blood cells, liver, testes, ovaries, colon, rectum, skin, stomach, esophagus, spleen, lymph nodes, bone marrow and kidney.

In yet a further embodiment, there is provided a method of diagnosing colorectal carcinoma comprising the steps of (i) obtaining a sample from a subject; and (ii) determining the loss or alteration of a functional BMPR1A gene in cells of the sample. The sample may be tissue or fluid, such as blood, buccal smear or amniocentesis sample. Determining may comprise assaying for a BMPR1A nucleic acid from the sample, for example, by amplifying the nucleic acid. The determining may also comprise contacting the sample with an antibody that binds immunologically to a BMPR1A, for example, in an ELISA.

The method may further comprise the step of comparing the expression of BMPR1A in the sample with the expression of BMPR1A in non-colorectal cancer samples. The comparison may involve evaluating the level of BMPR1A expression or evaluating the structure of the BMPR1A gene, protein or transcript. Evaluating may include an assay selected from the group consisting of sequencing, wild-type oligonucleotide hybridization, mutant oligonucleotide hybridization, SSCP, PCR™, denaturing gradient gel electrophoresis, antibody binding and RNase protection. In particular, evaluating comprises wild-type or mutant oligonucleotide hybridization, for example, using an oligonucleotide array on a chip or wafer. The colorectal carcinoma sample may comprise a mutation in the coding sequence of BMPR1A. The mutation may be a deletion, an insertion, a frameshift, a nonsense mutation, a missense mutation or splice mutation.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D: BMPR1A sequence variants in four JP kindreds. Top strand, wild-type sequence; bottom strand, mutant sequence; box surrounding, area of change. FIG. 1A, four-bp deletion in the D kindred. FIG. 1B, substitution in the E kindred. FIG. 1C, substitution in the B kindred. FIG. 1D, one-bp deletion in the S kindred.

FIGS. 2A-2D: Mutation testing in four JP kindreds. Black, known affected individuals; ?, at-risk individuals of all ages who have not been diagnosed with JP; arrows, bands corresponding to mutations. FIG. 2A, denaturing gel of exon 1 in the D kindred. FIG. 2B, single-strand conformation polymorphism (SSCP) gel of exon 7 in the E kindred. FIG. 2C, SSCP gel of exon 7 in the B kindred. FIG. 2D, denaturing gel of exon 8 in the S kindred.

### DESCRIPTION OF ILLUSTRATIVE EMBODIMENTS

Familial juvenile polyposis (JP) is a hamartomatous polyposis syndrome in which affected family members develop upper and lower gastrointestinal juvenile polyps and are at increased risk for gastrointestinal cancer. Other hamartomatous polyposis syndromes include Peutz-Jegher's Syndrome (PJS) and Cowden's Disease (CD). The genes for PJS and CD have been mapped to chromosome 19p (Hemminki et al., 1997; Hemminki et al., 1998; Mehenni et al., 1997), and 10q respectively, (Nelen et al., 1996; Liaw et al., 1997). A third entity, termed the “hereditary mixed-polyposis syndrome” (HMPS), differs from PJS and CD syndromes in that affected family members have a typical juvenile polyps, colonic adenomas, and colorectal carcinomas. A gene for HMPS has been mapped to chromosome 6q by linkage (Thomas et al., 1996), and it remains uncertain whether HMPS is a distinct clinical syndrome or a variant of FJP (Whitelaw et al., 1997).

Linkage studies in FJP families have resulted in the exclusion of APC and MCC as the genes for FJP (Leggett et al., 1993). Other genetic studies (Jacoby et al., 1997b), focused on the region of the PTEN gene, have shown that there is some evidence for a tumor-suppressor gene on 10q for FJP (termed “JP1”) (Jacoby et al., 1997a), but a recent study of 14 JP families found neither mutations in PTEN nor evidence of linkage to markers on 10q22-24 (Marsh et al., 1997). Analysis of an additional 11 cases of JP also did not uncover mutations in the PTEN gene (Riggins et al., 1997). Additionally, it appears that the clinical manifestation of polyps in patients with PTEN mutations are actually more likely to be attributable to CD rather than JP (Lynch et al., 1997; Olschwang et al., 1998). The present inventor previously provided linkage data that pointed to chromosome 18q21, and in 1998, he showed that a subset of juvenile polyposis families carry germ line mutations in the gene SMAD4 (also known as DPC4), located on chromosome 18q21.1. Howe et al. (1998b). However, SMAD4 mutations only occur in about 20% of JP cases, leaving many other cases unassigned.

1. The Present Invention

A particular objective of the present invention was to identify by linkage analysis an additional chromosomal locus for the JP gene in kindreds with generalized juvenile polyposis and gastrointestinal cancer. A similar study, previously undertaken by the inventor, identified the SMAD4 (DPC4) gene as being linked to the development of JP and gastrointestinal cancer. This new study shows, for the first time, that a previously identified gene, BMPR1A, also contributes to JP. This gene was first reported by ten Dijke et al. (1993), and designated ALK-3, who identified it as a novel activin receptor-like cell kinase having predicted serine/threonine activity. The results presented here confirm an important role for BMPR1A in the development of JP and gastrointestinal tumors. Methods and compositions for the diagnosis and treatment of such disorders, relying on the BMPR1A gene and protein, are presented herein below.

2. BMPR1A

BMPR1A (also knows as ALK-3) is a type I receptor of the TGF-β superfamily, with a cysteine-rich extracellular region, an intracellular glycine-serine-rich (GS) domain near the plasma membrane and an intracellular kinase domain (Heldin et al., 1997; Genbank Accession No. NM_{—}004329). There is a broad range of ligands in the BMP family, including BMPs 2-11, and these bind to specific type II BMP receptors. The type II receptors bind these ligands and activate the type I receptors through phosphorylation of their GS domain (Massague, 1996). When BMPR1A is activated through phosphorylation by the type II receptor, it then phosphorylates SMAD1 (Kretzschmar et al., 1997; Hoodless et al., 1996; Liu et al., 1996), SMAD5 and possibly SMAD8 (Heldin et al., 1997), which then associate with cytoplasmic SMAD4 (Kretzschmar et al., 1997; Lagna et al., 1996). These SMAD4-SMAD1, -5 or -9 complexes then migrate to the nucleus, associate with DNA-binding proteins and regulate the transcription of DNA sequences (Kretzschmar et al., 1997). See also, U.S. Pat. No. 6,207,814.

Nonsense mutations reported in four JP kindreds encode BMP receptors that lack the intracellular serine-threonine kinase domain (ten Dijke et al., 1993), and are predicted to result in loss of BMP-mediated intracellular signaling. The finding that germline mutations in both SMAD4 and BMPR1A result in the JP phenotype raises the question of whether the effects of SMAD4 are mediated through alterations in BMP signaling rather than through other TGF-β family members. In addition to their consequences for the diagnosis and management of families with JP, these results provide the first genetic evidence that BMPs are important to the control of epithelial neoplasia.

3. Juvenile Polyposis and Gastrointestinal Malignancy

The inherited polyposis syndromes can be divided into the adenomatous (FAP) and hamartomatous types, the latter of which includes familial juvenile polyposis (JP), Peutz-Jeghers Syndrome (PJ), and Cowden Disease (CD). The polyps in PJ patients are true hamartomata, but these may undergo adenomatous change and evolve into gastrointestinal malignancies. In CD, family members may develop multiple hamartomata of the skin, breast, thyroid, oral mucosa, or GI tract, and these individuals are at risk for breast and thyroid malignancies. Of these three syndromes, however, the strongest predisposition to gastrointestinal malignancy is seen in JP.

JP is an autosomal dominant condition characterized by juvenile polyps of the stomach small intestine, and/or colon in affected family members. It has been suggested that there are actually three forms of JP, with polyps developing in the colon, the stomach, or generalized throughout the GI tract (Goodman et al., 1979). It is also possible that these entities represent variable expressivity or different alterations of the same gene (Rustigi, 1994). In the past, many patients with JP presented with advanced cancers of the gastrointestinal tract, but now most are diagnosed by endoscopy following episodes of gastrointestinal bleeding, usually occurring within the first two decades of life. Surgical specimens from JP patients reveal multiple juvenile polyps, which have a unique microscopic appearance. Upon cursory inspection these polyps appear hyperplastic, but closer examination reveals dilated submucosal cystic spaces lined with glandular epithelium, an overabundance of stromal tissue, and infiltration of inflammatory cells.

In contrast to the relatively rare familial form of juvenile polyposis, sporadic juvenile polyps are the most common type of polyps seen in children, and may occur in 1-2% of the population (Jarvinen, 1993). These sporadic polyps do not predispose to malignancy, are usually solitary, slough at an early age and generally do not recur. Based upon the benign course of patients with sporadic juvenile polyps, polyps in patients with familial JP were originally thought to be hamartomata without malignant potential. However, in 1975, investigators at the University of Iowa described a kindred in which 10 family members had been diagnosed with juvenile polyposis of the upper and/or lower gastrointestinal (GI) tract, and 11 members had developed GI carcinomas (Stemper et al., 1975). Since this time, there have been many reports of GI malignancy developing in patients with juvenile polyposis, including colon cancer (Goodman et al., 1979; Stemper et al., 1975; Liu et al., 1978; Rozen and Baratz, 1982; Ramaswamy et al., 1984; Jarvinen and Franssila, 1984; Baptist and Sabatini, 1985; Bentley et al., 1989; Scott-Conner et al., 1995), stomach cancer (Stemper et al., 1975; Scott-Conner et al., 1995; Yoshida et al., 1988), and pancreatic cancer (Stemper et al., 1975; Walpole and Cullity, 1989). The risk of developing GI malignancy in affected family members has been estimated to be anywhere from 9% (Jarvinen and Franssila, 1984) to as high as 68% (Jass, 1994). The progression to adenocarcinoma has been hypothesized to begin with an adenomatous focus within a juvenile polyp, which later becomes dysplastic, and finally undergoes malignant transformation (Goodman et al., 1979; Jarvinen and Franssila, 1984).

Thus, juvenile polyposis may be diagnosed when a relatively old and asymptomatic parent is screened colonoscopically and the smallest number of polyps found on this basis is five. These data allow a working definition of juvenile polyposis to be formulated: (1) more than five juvenile polyps of the colorectum; and/or (2) juvenile polyps throughout the gastrointestinal tract; and/or (3) any number of juvenile polyps with a family history of juvenile polyposis.

The strong association of gastrointestinal carcinoma with juvenile polyposis suggests that the germline mutations predisposing to these unusual polyps also may play an important role in the development of sporadic gastrointestinal cancers, and in particular, colorectal and gastric cancer. By using a unique resource namely those individuals with a heritable predisposition to these tumors, the present inventor has been able to identify a gene which predisposes an individual to gastrointestinal cancer. The autosomal dominant inheritance seen in these kindreds allowed for a molecular genetic approach for the chromosomal localization of the gene, and the detection of mutations in candidate gene which segregate with the disease phenotype.

The majority of the hamartomatous polyposis syndromes have been genetically mapped or their predisposing genes have been identified. The PJ gene has been mapped to chromosome 19p by comparative genomic hybridization and linkage (Hemminki et al., 1997), and germline mutations identified in the serine threonine kinase gene LKB1 (Hemminki et al., 1998). The gene for CD was localized to chromosome 10q22-23 by linkage (Nelen et al., 1996), and germline mutations in the PTEN gene were later described in affected family members (Liaw et al., 1997). A third entity, termed the hereditary mixed polyposis syndrome (HMPS), differs in that affected family members have polyps similar to (but distinct from) juvenile polyps, as well as colonic adenomas and colorectal carcinomas. A gene for HMPS has been mapped to chromosome 6q by linkage (Thomas et al., 1996), and it remains uncertain whether HMPS is a distinct clinical syndrome or a variant of FJP (Whitelaw et al., 1997).

Linkage studies in JP families have excluded APC and MCC as the genes for JP (Leggett et al., 1993). Other genetic studies in JP have focused on the CD locus region on 10 q, stimulated by the finding of an interstitial deletion at 10q22-q24 in an infant with multiple colonic juvenile polyps and several congenital abnormalities (Jacoby et al., 1997a). Evaluation of juvenile polyps for loss of heterozygosity in this region revealed somatic deletions within the lamina propria in 39 of 47 polyps (83%) derived from 13 unrelated patients with FJP and 3 with sporadic juvenile polyps. These findings have suggested the presence of a tumor suppressor gene on 10q involved in JP (termed JP1) (Jacoby et al., 1997b). A recent report described 3 patients with juvenile polyposis who had germline mutations in the PTEN gene, but none were described as having a family history of juvenile polyposis (Olschwang et al., 1998). Another recent study of 14 JP families found neither mutations in PTEN nor evidence of linkage to markers on 10q22-24 (Marsh et al., 1997).

Linkage of the JP gene to markers on chromosome 18q21 in the kindred originally described by Stemper et al. (1975) also has been reported by the inventor in a previous study (Howe et al., 1998a). These data suggest genetic heterogeneity for the juvenile polyposis syndromes, and for the large family predisposed to GI carcinoma, linkage to the same region commonly deleted in sporadic colorectal (Fearon et al., 1990) and pancreatic carcinoma (Hahn et al., 1996). Although the gene involved in colorectal cancer was originally thought to be DCC (Fearon et al., 1990), subsequent studies have not provided compelling proof of mutations within the DCC gene.

The association of gastrointestinal cancers with JP, and the genetic linkage of the JP gene to the same interval on chromosome 18q21 frequently deleted in colorectal and pancreatic carcinomas suggests that identification of the JP gene could lend significant insight into the molecular mechanisms involved in these cancers. The inventor has further shown that a subset of JP families carry germline mutations in DPC4, also known as SMAD4. The mutant DPC4 proteins are predicted to be truncated at the carboxyl-terminus and lack sequences required for normal function. These results confirmed an important role for DPC4 in the development of JP and gastrointestinal tumors (Howe et al., 1998b).

4. Diagnosing Malignancy Involving BMPR1A

The present inventor has determined that alterations in BMPR1A are associated with the formation of juvenile polyposis (JP). Further, it is known that juvenile polyposis predisposes an individual to gastrointestinal malignancy as described herein above. Therefore, BMPR1A and the corresponding gene may be employed as a diagnostic or prognostic indicator of JP in general, and more particularly, of familial JP. More specifically, point mutations, deletions, insertions or regulatory perturbations relating to BMPR1A cause JP and/or promote cancer development, cause or promote polyposis or tumor progression at a primary site, and/or cause or promote metastasis.

The present invention contemplates further the diagnosis of colorectal cancer by detecting changes in the levels of BMPR1A expression. In one embodiment, BMPR1A immunostaining of colorectal samples utilizing an antibody recognizing the C-terminal end of the BMPR1A polypeptide, demonstrates a highly significant tendency for loss of BMPR1A expression during colorectal tumorigenesis.

In particular aspects, juvenile polyposis may be diagnosed using a combinantion of factors: (1) more than five juvenile polyps of the colorectum; and/or (2) juvenile polyps throughout the gastrointestinal tract; and/or (3) any number of juvenile polyps with a family history of juvenile polyposis; and/or (4) the presence of a mutated BMPR1A gene and a family history of juvenile polyposis.

I. Genetic Diagnosis

One embodiment of the present invention comprises a method for detecting variation in the expression of BMPR1A. This may comprise determining the level of BMPR1A or determining specific alterations in the expressed product. Obviously, this sort of assay has importance in the diagnosis of related cancers. Such cancer may involve cancers of the brain (glioblastomas, medulloblastoma, astrocytoma, oligodendroglioma, ependymomas), lung, liver, spleen, kidney, pancreas, bile ducts, ampulla of Vater, small intestine, blood cells, lymph nodes, colon, rectum, breast, endometrium, stomach, prostate, testicle, ovary, skin, head and neck, esophagus, bone marrow, blood or other tissue. In particular, the present invention relates to the diagnosis of juvenile polyposis which may or may not ultimately lead to gastrointestinal cancer.

The biological sample can be any tissue or fluid. Various embodiments include cells of the skin, muscle, fascia, brain, prostate, breast, endometrium, lung, head & neck, pancreas, small intestine, blood cells, liver, testes, ovaries, colon, rectum, skin, stomach, esophagus, spleen, lymph nodes, bone marrow or kidney. Other embodiments include fluid samples such as peripheral blood, lymph fluid, ascites, serous fluid, pleural effusion, sputum, cerebrospinal fluid, lacrimal fluid, stool urine or amniotic fluid.

Nucleic acids used are isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA (cDNA). In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Following detection, one may compare the results seen in a given patient with a statistically significant reference group of normal patients and patients that have BMPR1A-related pathologies. In this way, it is possible to correlate the amount or kind of BMPR1A detected with various clinical states.

Various types of defects have been identified by the present inventors. Thus, “alterations” should be read as including deletions, insertions, point mutations and duplications. Point mutations result in stop codons, frameshift mutations or amino acid substitutions. Somatic mutations are those occurring in non-germline tissues. Germ-line tissue can occur in any tissue and are inherited. Mutations in and outside the coding region also may affect the amount of BMPR1A produced, both by altering the transcription of the gene or in destabilizing or otherwise altering the processing of either the transcript (mRNA) or protein.

Many of the genes predisposing to the development of cancer are tumor suppressor genes, and conform to the two-hit hypothesis as described by Knudson for retinoblastoma (Knudson et al., 1975). Affected individuals from families with germline mutations in a tumor suppressor gene are born with a predisposition to cancer in all cells owing to the germline defect, and tumors develop after mutation or deletion of the remaining normal copy of the gene in a somatic cell. This results in loss of a functional protein whose role normally is to hold neoplastic transformation in check within the cell. Since many of these somatic events are deletions, mapping of these deletions helps to establish the minimal common region of overlap, which defines the region containing the tumor suppressor gene. Thus, a cell takes a genetic step toward oncogenic transformation when one allele of a tumor suppressor gene is inactivated due to inheritance of a germline lesion or acquisition of a somatic mutation. The inactivation of the other allele of the gene usually involves a somatic micromutation or chromosomal allelic deletion that results in loss of heterozygosity (LOH). Alternatively, both copies of a tumor suppressor gene may be lost by homozygous deletion.

In the D kindred, a four-bp deletion was detected in exon 1 (44-47delTGTT) of BMPR1A, resulting in a stop codon at nucleotides 104-106. In the E kindred, there was a transition at nucleotide 715, changing codon 239 from a glutamine to a stop codon (Q239X). In the B kindred, there was a transition at nucleotide 812, changing a tryptophan to a stop codon (W271X). In the S kindred, there was a one-bp deletion in exon 8 (961delC), creating a stop at the next codon.

All eight affected members of the D kindred had the four-bp deletion, as did one individual at risk (43 years of age). The inventors failed to identify this mutation in four other family members at risk without a diagnosis of JP (5-51 years of age). All eight affected members of the E kindred had the substitution in exon 7, whereas none of five family members (41-71 years of age) with-out JP had the mutation. In the B kindred, all five affected family members had the substitution in exon 7, as did one (35 years of age) of four family members without a diagnosis of JP (8-70 years of age). In the S kindred, all five affected kindred members had the deletion in exon 8, and this mutation was not found in any of the six family members without JP (19-77 years of age). The inventor did not find any of the four mutations described here in 250 normal control individuals. A polymorphism at nucleotide 4 (4A→C) was identified, and in 109 unrelated individuals, allele frequencies were 12% for the A/A genotype, 35% for A/C and 53% for C/C. Accordingly, this encoded for threonine at amino acid 2 (ACT, as in the published cDNA sequence) (ten Dijke et al., 1993) in 29% of chromosomes, and for proline (CCT) in 71%.

The existence of a locus for JP on 10q22-23 was first indicated by loss of heterozygosity (LOH) studies of sporadic and familial juvenile polyps. These studies documented deletions mapping to a 3-cM interval between D10S219 and D10S1696 (Jacoby et al., 1997b), 2.68 cM centromeric to D10S573 on the Center for Medical Genetics sex-averaged map. The gene PTEN, which predisposes to CS, was subsequently mapped to 10q22-23 (Nelen et al., 1996; Liaw et al., 1997). There has been confusion regarding whether JP may also be caused by mutations in PTEN (Eng et al., 1998). However, mutations in PTEN have not been identified in standard JP families, and PTEN was excluded as the JP gene in these families. Moreover, the discovery of germline nonsense mutations in BMPR1A provides compelling evidence that this is the gene on chromosome 10q22-23 responsible for JP in the families studied here. Thus, there are two closely linked genes on chromosome 10q22-23 that can cause hamartomatous polyps, but these genes have no apparent functional relationship and the extra-intestinal phenotypes associated with mutations in these genes are distinct.

It has been assumed that polyps develop in JP patients through a tumor-suppressor mechanism. After an individual with JP was found to have an interstitial deletion of 10q22-24, examination of markers from 10q22-23 for LOH in juvenile polyps demonstrated some degree of LOH in 39 of 47 juvenile polyps from 16 patients (Jacoby et al., 1997b). Fluorescent in situ hybridization analysis indicated that these deletions were specific to lymphocytes and macrophages in the lamina propria rather than in epithelial cells (Jacoby et al., 1 997b). Other studies have determined that deletions occur in the epithelium and fibroblasts of juvenile polyps, and have suggested that these cells may have a common clonal origin (Woodford-Richens et al., 2000).

The inventor assessed LOH of 10q markers in both epithelial and stromal fractions from six juvenile polyps from four patients after laser-capture microdissection. Although an occasional epithelial or stromal fraction showed partial LOH, no distinct or consistent result emerged. Either the cells in the polyps examined did not undergo LOH or there was an admixture of cell types with varying clonal origin in microdissected fractions, which prevented finding of LOH. This may be more definitively addressed in the future by in situ analysis using antibodies against the carboxyl terminus of BMPR1 A.

The mutations observed by the present inventors are listed in Table 1, which specifies the type of mutation, the exact nucleotide change, and the subsequent result of the mutation. Under the Type column, Fam stands for Familial, Spor stands for Sporadic, and Unk is Unknown. The type for JP44 is noted with a question mark as it is not clear whether or not it is Sporadic.

TABLE 1

| Case | Type | Exon | Nucleotide Change | Result |
|---|---|---|---|---|
| JP16 | Fam | 1 | 44-47delTGTT | stop 35-36 |
| JP19 | Fam | 2 | 184T > G | Y62D |
| JP59 | Spor | 3 | 245G > A | C82Y |
| JP24 | Fam | 3 | 262G > T | E84X |
| JP52 | Fam | 4 | 349C > T | Q117X |
| JP12 | Fam | 4 | 353delT | stop 122-123 |
| JP54 | Unk | 6 | 674delT | stop 259-260 |
| JP15 | Fam | 7 | 715C > T | Q239X |
| JP34 | Fam | 7 | 812G > A | W271X |
| JP60 | Spor | 7 | 864-868delACTTG | loss of exon 7 splice site |
| | | | IVS7 + 1-2delgt | |
| JP14 | Fam | 8 | 961delC | stop 321-322 |
| JP44 | Spor? | 8 | 1013C > A | A338D |
| JP7 | Fam | 8 | 1061delG | stop 363-364 |

It is contemplated that still other mutations in the BMPR1A gene may be identified in accordance with the present invention by detecting a nucleotide change in particular nucleic acids (U.S. Pat. No. 4,988,617, incorporated herein by reference). A variety of different assays are contemplated in this regard, including but not limited to, fluorescent in situ hybridization (FISH; U.S. Pat. No. 5,633,365 and U.S. Pat. No. 5,665,549, each incorporated herein by reference), direct DNA sequencing, PFGE analysis, Southern or Northern blotting, single-stranded conformation analysis (SSCA), RNAse protection assay, allele-specific oligonucleotide (ASO e.g., U.S. Pat. No. 5,639,611), dot blot analysis, denaturing gradient gel electrophoresis (e.g. U.S. Pat. No. 5,190,856 incorporated herein by reference), RFLP (e.g., U.S. Pat. No. 5,324,631 incorporated herein by reference) and PCR™-SSCP. Methods for detecting and quantitating gene sequences, such as mutated genes and oncogenes, in for example biological fluids are described in U.S. Pat. No. 5,496,699, incorporated herein by reference.

a. Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process.

In preferred embodiments, the probes or primers are labeled with radioactive species (³²P, ¹⁴C, ³⁵S, ³H, or other label), with a fluorophore (rhodamine, fluorescein) or a chemillumiscent (luciferase).

b. Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al., 1989. Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR” U.S. Pat. Nos. 5,494,810, 5,484,699, EPO No. 320 308, each incorporated herein by reference). In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase an RNA-directed RNA polymerase, also may be used as still another amplification method in the present invention. In this method, a replicative sequence of RNA that has a region complementary to that of a target is added to a sample in the presence of an RNA polymerase. The polymerase will copy the replicative sequence that can then be detected. Similar methods also are described in U.S. Pat. No. 4,786,600, incorporated herein by reference, which concerns recombinant RNA molecules capable of serving as a template for the synthesis of complementary single-stranded molecules by RNA-directed RNA polymerase. The product molecules so formed also are capable of serving as a template for the synthesis of additional copies of the original recombinant RNA molecule.

An isothermal amplification method, in which restriction endonucleases and ligases are used to achieve the amplification of target molecules that contain nucleotide 5′-[alpha-thio]-triphosphates in one strand of a restriction site also may be useful in the amplification of nucleic acids in the present invention (Walker et al., 1992; U.S. Pat. No. 5,270,184 incorporated herein by reference). U.S. Pat. No. 5,747,255 (incorporated herein by reference) describes an isothermal amplification using cleavable oligonucleotides for polynucleotide detection. In the method described therein, separated populations of oligonucleotides are provided that contain complementary sequences to one another and that contain at least one scissile linkage which is cleaved whenever a perfectly matched duplex is formed containing the linkage. When a target polynucleotide contacts a first oligonucleotide cleavage occurs and a first fragment is produced which can hybridize with a second oligonucleotide. Upon such hybridization, the second oligonucleotide is cleaved releasing a second fragment that can, in turn, hybridize with a first oligonucleotide in a manner similar to that of the target polynucleotide.

Strand Displacement Amplification (SDA) is another method of carrying out isothermal amplification of nucleic acids which involves multiple rounds of strand displacement and synthesis, i.e., nick translation (e.g., U.S. Pat. Nos. 5,744,311; 5,733,752; 5,733,733; 5,712,124). A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present invention. In the former application, “modified” primers are used in a PCR™-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR™” (Frohman, 1990; Ohara et al., 1989; each herein incorporated by reference in their entirety).

Methods based on ligation of two (or more) oligonucleotides in the presence of nucleic acid having the sequence of the resulting “di-oligonucleotide”, thereby amplifying the di-oligonucleotide, also may be used in the amplification step of the present invention. Wu et al., (1989), incorporated herein by reference in its entirety.

c. Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

d. Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

e. Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al., 1989. For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, 1994). The present invention provides methods by which any or all of these types of analyses may be used. Using the sequences disclosed herein, oligonucleotide primers may be designed to permit the amplification of sequences throughout the BMPR1A gene that may then be analyzed by direct sequencing.

f. Kit Components

All the essential materials and reagents required for detecting and sequencing BMPR1A and variants thereof may be assembled together in a kit. This generally will comprise preselected primers and probes. Also included may be enzymes suitable for amplifying nucleic acids including various polymerases (RT, Taq, Sequenase™, etc.), deoxynucleotides and buffers to provide the necessary reaction mixture for amplification. Such kits also generally will comprise, in suitable means, distinct containers for each individual reagent and enzyme as well as for each primer or probe.

g. Design and Theoretical Considerations for Relative Quantitative RT-PCR™

Reverse transcription (RT) of RNA to cDNA followed by relative quantitative PCR™ (RT-PCR™) can be used to determine the relative concentrations of specific mRNA species isolated from patients. By determining that the concentration of a specific mRNA species varies, it is shown that the gene encoding the specific mRNA species is differentially expressed.

In PCR™, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR™ amplification is directly proportional to the starting concentration of the target before the reaction began. By determining the concentration of the amplified products of the target DNA in PCR™ reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR™ products and the relative mRNA abundances is only true in the linear range of the PCR™ reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR™ for a collection of RNA populations is that the concentrations of the amplified PCR™ products must be sampled when the PCR™ reactions are in the linear portion of their curves.

The second condition that must be met for an RT-PCR™ experiment to successfully determine the relative abundances of a particular mRNA species is that relative concentrations of the amplifiable cDNAs must be normalized to some independent standard. The goal of an RT-PCR™ experiment is to determine the abundance of a particular mRNA species relative to the average abundance of all mRNA species in the sample. In the experiments described below, mRNAs for β-actin, asparagine synthetase and lipocortin II were used as external and internal standards to which the relative abundance of other mRNAs are compared.

Most protocols for competitive PCR™ utilize internal PCR™ standards that are approximately as abundant as the target. These strategies are effective if the products of the PCR™ amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The above discussion describes theoretical considerations for an RT-PCR m assay for clinically derived materials. The problems inherent in clinical samples are that they are of variable quantity (making normalization problematic), and that they are of variable quality (necessitating the co-amplification of a reliable internal control, preferably of larger size than the target). Both of these problems are overcome if the RT-PCR™ is performed as a relative quantitative RT-PCR™ with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other studies may be performed using a more conventional relative quantitative RT-PCR™ assay with an external standard protocol. These assays sample the PCR™ products in the linear portion of their amplification curves. The number of PCR™ cycles that are optimal for sampling must be empirically determined for each target cDNA fragment. In addition, the reverse transcriptase products of each RNA population isolated from the various tissue samples must be carefully normalized for equal concentrations of amplifiable cDNAs. This consideration is very important since the assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR™ assays can be superior to those derived from the relative quantitative RT-PCR™ assay with an internal standard.

One reason for this advantage is that without the internal standard/competitor, all of the reagents can be converted into a single PCR™ product in the linear range of the amplification curve, thus increasing the sensitivity of the assay. Another reason is that with only one PCR™ product, display of the product on an electrophoretic gel or another display method becomes less complex, has less background and is easier to interpret.

h. Chip Technologies

Specifically contemplated by the present inventors are chip-based DNA technologies such as those described by Hacia et al., (1996) and Shoemaker et al., (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization. See also Pease et al., (1994); Fodor et al., (1991).

II. Immunodiagnosis

Antibodies can be used in characterizing the BMPR1A content of healthy and diseased tissues, through techniques such as ELISAs and Western blotting. This may provide a screen for the presence or absence of malignancy or as a predictor of future cancer.

The use of antibodies of the present invention, in an ELISA assay is contemplated. For example, anti-BMPR1A antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for DPC4 that differs the first antibody. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2 to about 4 hr, at temperatures preferably on the order of about 25° to about 27° C. Following incubation, the antisera-contacted surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The steps of various other useful immunodetection methods have been described in the scientific literature, such as, e.g., Nakamura et al., (1987; incorporated herein by reference). Immunoassays, in their most simple and direct sense, are binding assays. Certain preferred immunoassays are the various types of radioimmunoassays (RIA) and immunobead capture assay. Immunohistochemical detection using tissue sections also is particularly useful. However, it will be readily appreciated that detection is not limited to such techniques, and Western blotting, dot blotting, FACS analyses, and the like also may be used in connection with the present invention.

The antibody compositions of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

5. Methods for Screening Active Compounds

The present invention also contemplates the use of BMPR1A and active fragments, and nucleic acids coding therefor, in the screening of compounds for activity in either stimulating BMPR1A activity, overcoming the lack of BMPR1A or blocking the effect of a mutant BMPR1A molecule. These assays may make use of a variety of different formats and may depend on the kind of “activity” for which the screen is being conducted. Contemplated functional “read-outs” include binding to a compound, inhibition of binding to a substrate, ligand, receptor or other binding partner by a compound, kinase activity, inhibition or stimulation of cell-to-cell signaling, growth, metastasis, cell division, cell migration, soft agar colony formation, contact inhibition, invasiveness, angiogenesis, apoptosis, tumor progression or other malignant phenotype.

I. In Vitro Assays

In one embodiment, the invention is to be applied for the screening of compounds that bind to the BMPR1A molecule or fragment thereof. The polypeptide or fragment may be either free in solution, fixed to a support, expressed in or on the surface of a cell. Either the polypeptide or the compound may be labeled, thereby permitting determining of binding.

In another embodiment, the assay may measure the inhibition of binding of BMPR1A to a natural or artificial substrate or binding partner. Competitive binding assays can be performed in which one of the agents (BMPR1A, binding partner or compound) is labeled. Usually, the polypeptide will be the labeled species. One may measure the amount of free label versus bound label to determine binding or inhibition of binding.

Another technique for high throughput screening of compounds is described in WO 84/03564. Large numbers of small peptide test compounds are synthesized on a solid substrate, such as plastic pins or some other surface. The peptide test compounds are reacted with BMPR1A and washed. Bound polypeptide is detected by various methods.

Purified BMPR1A can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link the BMPR1A active region to a solid phase.

Various cell lines containing wild-type or natural or engineered mutations in BMPR1A can be used to study various functional attributes of BMPR1A and how a candidate compound affects these attributes. Methods for engineering mutations are described elsewhere in this document, as are naturally-occurring mutations in BMPR1A that lead to, contribute to and/or otherwise cause malignancy. In such assays, the compound would be formulated appropriately, given its biochemical nature, and contacted with a target cell. Depending on the assay, culture may be required. The cell may then be examined by virtue of a number of different physiologic assays. Alternatively, molecular analysis may be performed in which the function of BMPR1A, or related pathways, may be explored. This may involve assays such as those for protein expression, enzyme function, substrate utilization, phosphorylation states of various molecules including BMPR1A, cAMP levels, mRNA expression (including differential display of whole cell or polyA RNA) and others.

II. In Vivo Assays

The present invention also encompasses the use of various animal models. Thus, any identity seen between human and other animal BMPR1A provides an excellent opportunity to examine the function of BMPR1A in a whole animal system where it is normally expressed. By developing or isolating mutant cells lines that fail to express normal BMPR1A, one can generate models in mice that will be highly predictive of juvenile polyposis and related cancers in humans and other mammals. These models may employ the orthotopic or systemic administration of tumor cells to mimic juvenile polyposis and/or associated cancers. Alternatively, one may induce such a malignant phenotype in animals by providing agents known to be responsible for certain events associated with malignant transformation and/or tumor progression. Finally, transgenic animals (discussed below) that lack a wild-type BMPR1A may be utilized as models for juvenile polyposis and cancer development and treatment.

Treatment of animals with test compounds will involve the administration of the compound, in an appropriate form, to the animal. Administration will be by any route the could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intratumoral injection.

Determining the effectiveness of a compound in vivo may involve a variety of different criteria. Such criteria include, but are not limited to, survival, reduction of tumor burden or mass, arrest or slowing of tumor progression, elimination of tumors, inhibition or prevention of metastasis, increased activity level, improvement in immune effector function and improved food intake.

III. Rational Drug Design

The goal of rational drug design is to produce structural analogs of biologically active polypeptides or compounds with which they interact (agonists, antagonists, inhibitors, binding partners, etc.). By creating such analogs, it is possible to fashion drugs which are more active or stable than the natural molecules, which have different susceptibility to alteration or which may affect the function of various other molecules. In one approach, one would generate a three-dimensional structure for BMPR1A or a fragment thereof. This could be accomplished by x-ray crystallography, computer modeling or by a combination of both approaches. An alternative approach, “alanine scan,” involves the random replacement of residues throughout molecule with alanine, and the resulting affect on function determined.

It also is possible to isolate a BMPR1A specific antibody, selected by a functional assay, and then solve its crystal structure. In principle, this approach yields a pharmacore upon which subsequent drug design can be based. It is possible to bypass protein crystallograph altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Thus, one may design drugs which have improved BMPR1A activity or which act as stimulators, inhibitors, agonists, antagonists of BMPR1A or molecules affected by BMPR1A function. By virtue of the availability of cloned BMPR1A sequences, sufficient amounts of BMPR1A can be produced to perform crystallographic studies. In addition, knowledge of the polypeptide sequences permits computer employed predictions of structure-function relationships.

IV. Transgenic Animals/Knockout Animals

In one embodiment of the invention, transgenic animals are produced which contain a functional transgene encoding a functional BMPR1A polypeptide or variants thereof. Transgenic animals expressing BMPR1A transgenes, recombinant cell lines derived from such animals and transgenic embryos may be useful in methods for screening for and identifying agents that induce or repress function of BMPR1A. Transgenic animals of the present invention also can be used as models for studying indications such as cancers.

In one embodiment of the invention, a BMPR1A transgene is introduced into a non-human host to produce a transgenic animal expressing a human or murine BMPR1A gene. The transgenic animal is produced by the integration of the transgene into the genome in a manner that permits the expression of the transgene. Methods for producing transgenic animals are generally described by Wagner and Hoppe (U.S. Pat. No. 4,873,191; which is incorporated herein by reference), Brinster et al., 1985; which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

It may be desirable to replace the endogenous BMPR1A by homologous recombination between the transgene and the endogenous gene; or the endogenous gene may be eliminated by deletion as in the preparation of “knock-out” animals. Typically, a BMPR1A gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Within a particularly preferred embodiment, transgenic mice are generated which overexpress BMPR1A or express a mutant form of the polypeptide. Alternatively, the absence of a BMPR1A in “knock-out” mice permits the study of the effects that loss of BMPR1A protein has on a cell in vivo. Knock-out mice also provide a model for the development of BMPR1A-related malignancy for example, juvenile polyposis.

As noted above, transgenic animals and cell lines derived from such animals may find use in certain testing experiments. In this regard, transgenic animals and cell lines capable of expressing wild-type or mutant BMPR1A may be exposed to test substances. These test substances can be screened for the ability to enhance wild-type BMPR1A expression and or function or impair the expression or function of mutant BMPR1A.

6. Methods for Treating BMPR1A Related Malignancies

The present invention also involves, in another embodiment, the treatment of juvenile polyposis and cancer. The types of malignancy that may be treated, according to the present invention, is limited only by the involvement of BMPR1A. By involvement, it is not even a requirement that BMPR1A be mutated or abnormal—the overexpression of this tumor suppressor may actually overcome other lesions within the cell. Thus, it is contemplated that a wide variety of tumors may be treated using BMPR1A therapy, including cancers of the pancreas, small intestine, large intestine, colon, stomach, rectal tumors or other tissue.

In many contexts, it is not necessary that the tumor cell be killed or induced to undergo normal cell death or “apoptosis.” Rather, to accomplish a meaningful treatment, all that is required is that the tumor growth be slowed to some degree. It may be that the tumor growth is completely blocked, however, or that some tumor regression is achieved. Clinical terminology such as “remission” and “reduction of tumor” burden also are contemplated given their normal usage.

I. Genetic Based Therapies

One of the therapeutic embodiments contemplated by the present inventors is the intervention, at the molecular level, in the events involved in the tumorigenesis of some cancers. Specifically, the present inventors intend to provide, to a juvenile polyposis cell (or even a subsequent cancer cell), an expression construct capable of providing BMPR1A to that cell. Because the sequence homology between the human, and other BMPR1As, any of these nucleic acids could be used in human therapy, as could any of the gene sequence variants discussed above which would encode the same, or a biologically equivalent polypeptide. The lengthy discussion of expression vectors and the genetic elements employed therein is incorporated into this section by reference. Particularly preferred expression vectors are viral vectors such as adenovirus, adeno-associated virus, herpesvirus, vaccinia virus and retrovirus. Also preferred is liposomally-encapsulated expression vector.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

Various routes are contemplated for various tumor types. The section below on routes contains an extensive list of possible routes. For practically any tumor, systemic delivery is contemplated. This will prove especially important for attacking microscopic polyposis and microscopic cancer. Where discrete polyposis mass may be identified, a variety of direct, local and regional approaches may be taken. For example, the tumor may be directly injected with the expression vector. A polyp (or tumor) bed may be treated prior to, during or after resection. Following resection, one generally will deliver the vector by a catheter left in place following surgery. One may utilize the tumor vasculature to introduce the vector into the tumor by injecting a supporting vein or artery. A more distal blood supply route also may be utilized.

In a different embodiment, ex vivo gene therapy is contemplated. This approach is particularly suited, although not limited, to treatment of bone marrow associated cancers. In an ex vivo embodiment, cells from the patient are removed and maintained outside the body for at least some period of time. During this period, a therapy is delivered, after which the cells are reintroduced into the patient; hopefully, any malignant cells in the sample have been killed.

Autologous bone marrow transplant (ABMT) is an example of ex vivo gene therapy. Basically, the notion behind ABMT is that the patient will serve as his or her own bone marrow donor. Thus, a normally lethal dose of irradiation or chemotherapeutic may be delivered to the patient to kill malignant cells, and the bone marrow repopulated with the patients own cells that have been maintained (and perhaps expanded) ex vivo. Because, bone marrow often is contaminated with tumor cells, it is desirable to purge the bone marrow of these cells. Use of gene therapy to accomplish this goal is yet another way BMPR1A may be utilized according to the present invention.

II. Immunotherapies

Immunotherapeutics, generally, rely on the use of immune effector cells and molecules to target and destroy malignant cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

According to the present invention, it is unlikely that BMPR1A could serve as a target for an immune effector given that (i) it is unlikely to be expressed on the surface of the cell and (ii) that the presence, not absence, of BMPR1A is associated with the normal state. However, it is possible that particular mutant forms of BMPR1A may be targeted by immunotherapy, either using antibodies, antibody conjugates or immune effector cells.

A more likely scenario is that immunotherapy could be used as part of a combined therapy, in conjunction with BMPR1A-targeted gene therapy. The general approach for combined therapy is discussed below. Generally, the tumor cell must bear some marker that is amenable to targeting, i.e., is not present on the majority of other cells. Many tumor marker exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

The invention further provides immunotoxins in which an antibody that binds to a cancer marker, such as a mutant BMPR1A, is linked to a cytotoxic agent. Immunotoxin technology is fairly well-advanced and known to those of skill in the art. Immunotoxins are agents in which the antibody component is linked to another agent, particularly a cytotoxic or otherwise anticellular agent, having the ability to kill or suppress the growth or cell division of cells.

As used herein, the terms “toxin” and “toxic moiety” are employed to refer to any cytotoxic or otherwise anticellular agent that has such a killing or suppressive property. Toxins are thus pharmacologic agents that can be conjugated to an antibody and delivered in an active form to a cell, wherein they will exert a significant deleterious effect.

The preparation of immunotoxins is, in general, well known in the art (see, e.g., U.S. Pat. No. 4,340,535, incorporated herein by reference). It also is known that while IgG based immunotoxins will typically exhibit better binding capability and slower blood clearance than their Fab′ counterparts, Fab′ fragment-based immunotoxins will generally exhibit better tissue penetrating capability as compared to IgG based immunotoxins.

Exemplary anticellular agents include chemotherapeutic agents, radioisotopes as well as cytotoxins. Example of chemotherapeutic agents are hormones such as steroids; antimetabolites such as cytosine arabinoside, fluorouracil, methotrexate or aminopterin; anthracycline; mitomycin C; vinca alkaloids; demecolcine; etoposide; mithramycin; or alkylating agents such as chlorambucil or melphalan.

Preferred immunotoxins often include a plant-, fungal- or bacterial-derived toxin, such as an A chain toxin, a ribosome inactivating protein, α-sarcin, aspergillin, restirictocin, a ribonuclease, diphtheria toxin or pseudomonas exotoxin, to mention just a few examples. The use of toxin-antibody constructs is well known in the art of immunotoxins, as is their attachment to antibodies. Of course, combinations of the various toxins could also be coupled to one antibody molecule, thereby accommodating variable or even enhanced cytotoxicity.

One type of toxin for attachment to antibodies is ricin, with deglycosylated ricin A chain being particularly preferred. As used herein, the term “ricin” is intended to refer to ricin prepared from both natural sources and by recombinant means. Various recombinant or genetically engineered forms of the ricin molecule are known to those of skill in the art, all of which may be employed in accordance with the present invention.

Deglycosylated ricin A chain (dgA) is preferred because of its extreme potency, longer half-life, and because it is economically feasible to manufacture it a clinical grade and scale (available commercially from Inland Laboratories, Austin, Tex.). Truncated ricin A chain, from which the 30 N-terminal amino acids have been removed by Nagarase (Sigma), also may be employed.

Linking or coupling one or more toxin moieties to an antibody may be achieved by a variety of mechanisms, for example, covalent binding, affinity binding, intercalation, coordinate binding and complexation. Preferred binding methods are those involving covalent binding, such as using chemical cross-linkers, natural peptides or disulfide bonds.

The covalent binding can be achieved either by direct condensation of existing side chains or by the incorporation of external bridging molecules. Many bivalent or polyvalent agents are useful in coupling protein molecules to other proteins, peptides or amine functions. Examples of coupling agents are carbodiimides, diisocyanates, glutaraldehyde, diazobenzenes, and hexamethylene diamines. This list is not intended to be exhaustive of the various coupling agents known in the art but, rather, is exemplary of the more common coupling agents that may be used.

In preferred embodiments, it is contemplated that one may wish to first derivatize the antibody, and then attach the toxin component to the derivatized product. As used herein, the term “derivatize” is used to describe the chemical modification of the antibody substrate with a suitable cross-linking agent. Examples of cross-linking agents for use in this manner include the disulfide-bond containing linkers SPDP (N-succinimidyl-3-(2-pyridyldithio)propionate) and SMPT (4-succinimidyl-oxycarbonyl-α-methyl-α(2-pyridyldithio)toluene).

Biologically releasable bonds are particularly important to the realization of a clinically active immunotoxin in that the toxin moiety must be capable of being released from the antibody once it has entered the target cell. Numerous types of linking constructs are known, including simply direct disulfide bond formation between sulfhydryl groups contained on amino acids such as cysteine, or otherwise introduced into respective protein structures, and disulfide linkages using available or designed linker moieties.

Numerous types of disulfide-bond containing linkers are known which can successfully be employed to conjugate toxin moieties to antibodies, however, certain linkers are generally preferred, such as, for example, sterically hindered disulfide bond linkers are preferred due to their greater stability in vivo, thus preventing release of the toxin moiety prior to binding at the site of action. A particularly preferred cross-linking reagent is SMPT, although other linkers such as SATA, SPDP and 2-iminothiolane also may be employed.

Once conjugated, it will be important to purify the conjugate so as to remove contaminants such as unconjugated A chain or antibody. It is important to remove unconjugated A chain because of the possibility of increased toxicity. Moreover, it is important to remove unconjugated antibody to avoid the possibility of competition for the antigen between conjugated and unconjugated species. In any event, a number of purification techniques have been found to provide conjugates to a sufficient degree of purity to render them clinically useful.

In general, the most preferred technique will incorporate the use of Blue-Sepharose with a gel filtration or gel permeation step. Blue-Sepharose is a column matrix composed of Cibacron Blue 3GA and agarose, which has been found to be useful in the purification of immunoconjugates. The use of Blue-Sepharose combines the properties of ion exchange with A chain binding to provide good separation of conjugated from unconjugated binding. The Blue-Sepharose allows the elimination of the free (non conjugated) antibody from the conjugate preparation. To eliminate the free (unconjugated) toxin (e.g., dgA) a molecular exclusion chromatography step may be used using either conventional gel filtration procedure or high performance liquid chromatography.

After a sufficiently purified conjugate has been prepared, one will generally desire to prepare it into a pharmaceutical composition that may be administered parenterally. This is done by using for the last purification step a medium with a suitable pharmaceutical composition. Such formulations will typically include pharmaceutical buffers, along with excipients, stabilizing agents and such like. The pharmaceutically acceptable compositions will be sterile, non-immunogenic and non-pyrogenic. Details of their preparation are well known in the art and are further described herein. It will be appreciated that endotoxin contamination should be kept minimally at a safe level, for example, less that 0.5 ng/mg protein.

Suitable pharmaceutical compositions in accordance with the invention will generally comprise from about 10 to about 100 mg of the desired conjugate admixed with an acceptable pharmaceutical diluent or excipient, such as a sterile aqueous solution, to give a final concentration of about 0.25 to about 2.5 mg/ml with respect to the conjugate.

As mentioned above, the antibodies of the invention may be linked to one or more chemotherapeutic agents, such as anti-tumor drugs, cytokines, antimetabolites, alkylating agents, hormones, nucleic acids and the like, which may thus be targeted to a BMPR1A expressing cell using the antibody conjugate. The advantages of antibody-conjugated agents over their non-antibody conjugated counterparts is the added selectivity afforded by the antibody.

In analyzing the variety of chemotherapeutic and pharmacologic agents available for conjugating to an antibody, one may wish to particularly consider those that have been previously shown to be successfully conjugated to antibodies and to function pharmacologically. Exemplary antineoplastic agents that have been used include doxorubicin, daunomycin, methotrexate, vinblastine. Moreover, the attachment of other agents such as neocarzinostatin, macromycin, trenimon and α-amanitin has also been described. The lists of suitable agents presented herein are, of course, merely exemplary in that the technology for attaching pharmaceutical agents to antibodies for specific delivery to tissues is well established.

Thus, it is generally believed to be possible to conjugate to antibodies any pharmacologic agent that has a primary or secondary amine group, hydrazide or hydrazine group, carboxyl alcohol, phosphate, or alkylating group available for binding or cross-linking to the amino acids or carbohydrate groups of the antibody. In the case of protein structures, this is most readily achieved by means of a cross linking agent, as described above for the immunotoxins. Attachment also may be achieved by means of an acid labile acyl hydrazone or cis aconityl linkage between the drug and the antibody, or by using a peptide spacer such as L-Leu-L-Ala-L-Leu-L-Ala, between the γ-carboxyl group of the drug and an amino acid of the antibody.

III. Protein Therapy

Another therapy approach is the provision, to a subject, of BMPR1A polypeptide, active fragments, synthetic peptides, mimetics or other analogs thereof. The protein may be produced by recombinant expression means or, if small enough, generated by an automated peptide synthesizer. Formulations would be selected based on the route of administration and purpose including, but not limited to, liposomal formulations and classic pharmaceutical preparations.

IV. Combined Therapy with Immunotherapy, Traditional Chemo- or Radiotherapy

Tumor cell resistance to DNA damaging agents represents a major problem in clinical oncology. One goal of current cancer research is to find ways to improve the efficacy of chemo- and radiotherapy. One way is by combining such traditional therapies with gene therapy. For example, the herpes simplex-thymidine kinase (HS-tk) gene, when delivered to brain tumors by a retroviral vector system, successfully induced susceptibility to the antiviral agent ganciclovir (Culver et al., 1992). In the context of the present invention, it is contemplated that BMPR1A replacement therapy could be used similarly in conjunction with chemo- or radiotherapeutic intervention. It also may prove effective to combine BMPR1A gene therapy with immunotherapy, as described above.

To kill cells, inhibit cell growth, inhibit metastasis, inhibit angiogenesis or otherwise reverse or reduce the malignant phenotype of tumor cells, using the methods and compositions of the present invention, one would generally contact a “target” cell with a BMPR1A expression construct and at least one other agent. These compositions would be provided in a combined amount effective to kill or inhibit proliferation of the cell. This process may involve contacting the cells with the expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes the expression construct and the other includes the agent.

Alternatively, the gene therapy treatment may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either BMPR1A or the other agent will be desired. Various combinations may be employed, where BMPR1A is “A” and the other agent is “B”, as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are contemplated. Again, to achieve cell killing, both agents are delivered to a cell in a combined amount effective to kill the cell.

Agents or factors suitable for use in a combined therapy are any chemical compound or treatment method that induces DNA damage when applied to a cell. Such agents and factors include radiation and waves that induce DNA damage such as, γ-irradiation, X-rays, UV-irradiation, microwaves, electronic emissions, and the like. A variety of chemical compounds, also described as “chemotherapeutic agents,” function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP) and even hydrogen peroxide. The invention also encompasses the use of a combination of one or more DNA damaging agents, whether radiation-based or actual compounds, such as the use of X-rays with cisplatin or the use of cisplatin with etoposide. In certain embodiments, the use of cisplatin in combination with a BMPR1A expression construct is particularly preferred as this compound.

In treating juvenile polyposis or a related cancer according to the invention, one would contact the tumor cells with an agent in addition to the expression construct. This may be achieved by irradiating the localized tumor site with radiation such as X-rays, UV-light, γ-rays or even microwaves. Alternatively, the tumor cells may be contacted with the agent by administering to the subject a therapeutically effective amount of a pharmaceutical composition comprising a compound such as, adriamycin, 5-fluorouracil, etoposide, camptothecin, actinomycin-D, mitomycin C, or more preferably, cisplatin. The agent may be prepared and used as a combined therapeutic composition, or kit, by combining it with a BMPR1A expression construct, as described above.

Agents that directly cross-link nucleic acids, specifically DNA, are envisaged to facilitate DNA damage leading to a synergistic, antineoplastic combination with BMPR1A. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/M² at 21 day intervals for adriamycin, to 35-50 mg/m² for etoposide intravenously or double the intravenous dose orally.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, X-rays, and/or the directed delivery of radioisotopes to tumor cells. Other forms of DNA damaging factors also are contemplated such as microwaves and UV-irradiation. It is most likely that all of these factors effect a broad range of damage DNA, on the precursors of DNA, the replication and repair of DNA, and the assembly and maintenance of chromosomes. Dosage ranges for X-rays range from daily doses of 50 to 200 gGy for prolonged periods of time (3 to 4 weeks), to single doses of 2000 to 6000 gGy. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventors propose that the regional delivery of BMPR1A expression constructs to patients with BMPR1A-linked cancers will be a very efficient method for delivering a therapeutically effective gene to counteract the clinical disease. Similarly, the chemo- or radiotherapy may be directed to a particular, affected region of the subjects body. Alternatively, systemic delivery of expression construct and/or the agent may be appropriate in certain circumstances, for example, where extensive metastasis has occurred.

In addition to combining BMPR1A-targeted therapies with chemo- and radiotherapies, it also is contemplated that combination with other gene therapies will be advantageous. For example, targeting of BMPR1A and p53 or p16 mutations at the same time may produce an improved anti-cancer treatment. Any other tumor-related gene conceivably can be targeted in this manner, for example, p21, Rb, APC, DCC, NF-1, NF-2, BCRA2, p16, FHIT, WT-1, MEN-1, RET, BRCA1, VHL, FCC, MCC, ras, myc, neu, raf erb, src, fms, jun, trk, ret, gsp, hst, bcl and abl.

It also should be pointed out that any of the foregoing therapies may prove useful by themselves in treating a mutant BMPR1A. In this regard, reference to chemotherapeutics and non-BMPR1A gene therapy in combination should also be read as a contemplation that these approaches may be employed separately.

6. Engineering Expression Constructs

In certain embodiments, the present invention involves the manipulation of genetic material to produce expression constructs that encode a therapeutic gene for the treatment of juvenile polyposis and/or related cancer. Such methods involve the generation of expression constructs containing, for example, a heterologous DNA encoding a gene of interest and a means for its expression, replicating the vector in an appropriate helper cell, obtaining viral particles produced therefrom, and infecting cells with the recombinant virus particles.

The gene will be a normal BMPR1A gene discussed herein above, or the gene may be a second therapeutic gene or nucleic acid useful in the treatment of, for example cancer cells. In the context of gene therapy, the gene will be a heterologous DNA, meant to include DNA derived from a source other than the viral genome which provides the backbone of the vector. Finally, the virus may act as a live viral vaccine and express an antigen of interest for the production of antibodies thereagainst. The gene may be derived from a prokaryotic or eukaryotic source such as a bacterium, a virus, a yeast, a parasite, a plant, or even an animal. The heterologous DNA also may be derived from more than one source, i.e., a multigene construct or a fusion protein. The heterologous DNA also may include a regulatory sequence which may be derived from one source and the gene from a different source.

I. Additional Therapeutic Genes

The present invention contemplates the use of a variety of different genes in combination with BMPR1A gene constructs. For example, genes encoding enzymes, hormones, cytokines, oncogenes, receptors, tumor suppressors, transcription factors, drug selectable markers, toxins and various antigens are contemplated as suitable genes for use according to the present invention. In addition, antisense constructs derived from oncogenes are other “genes” of interest according to the present invention.

As described herein above BMPR1A has now been shown to be mutated in JP. Its Genbank accession No. is NM004329, specifically incorporated herein by reference). In certain embodiments, of the present invention, it will be possible to introduce wild-type BMPR1A to juvenile polyposis cells.

a. Other Tumor Suppressors

The genetic constructs of the present invention may further comprise other tumor suppressor in combination with BMPR1A. p53 is one such ubiquitously recognized as a tumor suppressor gene (Hollstein et al., 1991; U.S. Pat. No. 5,747,469, specifically incorporated herein by reference in its entirety). Other tumor related genes that could be used herein include p16^{INK4} (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995); Cell adhesion molecules, or CAM's (Edelman and Crossin, 1991; Frixen et al., 1991; Bussemakers et al., 1992; Matsura et al., 1992; Umbas et al., 1992); RB, APC, DCC, NF-1, NF-2, WT-1, MEN-1, MEN-II, zacl, p73, VHL, MMAC1, FCC and MCC. Additionally, inducers f apoptosis also are contemplated for use in combination with BMPR1A, these include members of the Bcl-2 family (Bax, Bax, Bak, Bcl-X_{S}, Bik, Bid, Bad, Harakiri) as well as, Ad E1B and ICE-CED3 proteases, similarly could find use according to the present invention.

b. Enzymes

Various enzyme genes are of interest according to the present invention. Such enzymes include cytosine deaminase, hypoxanthine-guanine phosphoribosyltransferase, galactose-1-phosphate uridyltransferase, phenylalanine hydroxylase, glucocerbrosidase, sphingomyelinase, α-L-iduronidase, glucose-6-phosphate dehydrogenase, HSV thymidine kinase and human thymidine kinase.

c. Cytokines

Other classes of genes that are contemplated to be inserted into the therapeutic expression constructs of the present invention include interleukins and cytokines. Interleukin 1 (IL-1), IL-2, IL-3, IL-4, IL-5, IL-6, IL-7, IL-8, IL-9, IL-10, IL-11 IL-12, GM-CSF and G-CSF.

d. Antibodies

In yet another embodiment, the heterologous gene may include a single-chain antibody. Methods for the production of single-chain antibodies are well known to those of skill in the art. The skilled artisan is referred to U.S. Pat. No. 5,359,046, (incorporated herein by reference) for such methods. A single chain antibody is created by fusing together the variable domains of the heavy and light chains using a short peptide linker, thereby reconstituting an antigen binding site on a single molecule.

Single-chain antibody variable fragments (Fvs) in which the C-terminus of one variable domain is tethered to the N-terminus of the other via a 15 to 25 amino acid peptide or linker, have been developed without significantly disrupting antigen binding or specificity of the binding (Bedzyk et al., 1990; Chaudhary et al., 1990). These Fvs lack the constant regions (Fc) present in the heavy and light chains of the native antibody.

Antibodies to a wide variety of molecules can be used in combination with the present invention, including antibodies against oncogenes, toxins, hormones, enzymes, viral or bacterial antigens, transcription factors, receptors and the like.

II. Antisense constructs

In some embodiments, it may prove beneficial to inhibit a dominant negative BMPR1A product. Also, oncogenes such as ras, myc, neu, raf erb, src, fms, jun, trk, ret, gsp, hst, and abl as well as the antiapoptotic member of the Bcl-2 family, are suitable targets in combination with BMPR1A therapies. However, for therapeutic benefit, these oncogenes would be expressed as an antisense nucleic acid, so as to inhibit the expression of the oncogene. The term “antisense nucleic acid” is intended to refer to the oligonucleotides complementary to the base sequences of oncogene-encoding DNA and RNA. Antisense oligonucleotides, when introduced into a target cell, specifically bind to their target nucleic acid and interfere with transcription, RNA processing, transport and/or translation. Targeting double-stranded (ds) DNA with oligonucleotide leads to triple-helix formation; targeting RNA will lead to double-helix formation.

Antisense constructs may be designed to bind to the promoter and other control regions, exons, introns or even exon-intron boundaries of a gene. Antisense RNA constructs, or DNA encoding such antisense RNAs, may be employed to inhibit gene transcription or translation or both within a host cell, either in vitro or in vivo, such as within a host animal, including a human subject. Nucleic acid sequences comprising “complementary nucleotides” are those which are capable of base-pairing according to the standard Watson-Crick complementary rules. That is, that the larger purines will base pair with the smaller pyrimidines to form only combinations of guanine paired with cytosine (G:C) and adenine paired with either thymine (A:T), in the case of DNA, or adenine paired with uracil (A:U) in the case of RNA.

As used herein, the terms “complementary” or “antisense sequences” mean nucleic acid sequences that are substantially complementary over their entire length and have very few base mismatches. For example, nucleic acid sequences of fifteen bases in length may be termed complementary when they have a complementary nucleotide at thirteen or fourteen positions with only single or double mismatches. Naturally, nucleic acid sequences which are “completely complementary” will be nucleic acid sequences which are entirely complementary throughout their entire length and have no base mismatches.

While all or part of the gene sequence may be employed in the context of antisense construction, statistically, any sequence 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that oligonucleotides of 8, 9, 10, 11, 12, 13, 14, 15, 16, 17, 18, 19, 20 or more base pairs will be used. One can readily determine whether a given antisense nucleic acid is effective at targeting of the corresponding host cell gene simply by testing the constructs in vitro to determine whether the endogenous gene's function is affected or whether the expression of related genes having complementary sequences is affected.

In certain embodiments, one may wish to employ antisense constructs which include other elements, for example, those which include C-5 propyne pyrimidines. Oligonucleotides which contain C-5 propyne analogues of uridine and cytidine have been shown to bind RNA with high affinity and to be potent antisense inhibitors of gene expression (Wagner et al., 1993).

III. Ribozyme Constructs

Much as with antisense, ribozymes can be used in the present invention to attack dominant negative BMPR1A products. As an alternative to targeted antisense delivery, targeted ribozymes may be used. The term “ribozyme” refers to an RNA-based enzyme capable of targeting and cleaving particular base sequences in oncogene DNA and RNA. Ribozymes either can be targeted directly to cells, in the form of RNA oligo-nucleotides incorporating ribozyme sequences, or introduced into the cell as an expression construct encoding the desired ribozymal RNA. Ribozymes may be used and applied in much the same way as described for antisense nucleic acids.

IV. Selectable Markers

In certain embodiments of the invention, the therapeutic expression constructs of the present invention contain nucleic acid constructs whose expression may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin and histidinol are useful selectable markers. Alternatively, enzymes such as herpes simplex virus thymidine kinase (tk) may be employed. Immunologic markers also can be employed. The selectable marker employed is not believed to be important, so long as it is capable of being expressed simultaneously with the nucleic acid encoding a gene product. Further examples of selectable markers are well known to one of skill in the art and include reporters such as EGFP, β-gal or chloramphenicol acetyltransferase (CAT).

V. Multigene Constructs and IRES

In certain embodiments of the invention, the use of internal ribosome binding sites (IRES) elements are used to create multigene polycistronic messages. IRES elements are able to bypass the ribosome scanning model of 5′-methylated, Cap-dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

VI. Control Regions

a. Promoters

Throughout this application, the term “expression construct” is meant to include any type of genetic construct containing a nucleic acid coding for gene products in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding genes of interest.

The nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example in the case where expression of a transgene, or transgenes when a multicistronic vector is utilized, is toxic to the cells in which the vector is produced in, it may be desirable to prohibit or reduce expression of one or more of the transgenes. Examples of transgenes that may be toxic to the producer cell line are pro-apoptotic and cytokine genes. Several inducible promoter systems are available for production of viral vectors where the transgene product may be toxic.

The ecdysone system (Invitrogen, Carlsbad, Calif.) is one such system. This system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-responsive promoter which drives expression of the gene of interest is on another plasmid. Engineering of this type of system into the gene transfer vector of interest would therefore be useful. Cotransfection of plasmids containing the gene of interest and the receptor monomers in the producer cell line would then allow for the production of the gene transfer vector without expression of a potentially toxic transgene. At the appropriate time, expression of the transgene could be activated with ecdysone or muristeron A.

Another inducible system that would be useful is the Tet-Off™ or Tet-On™ system (Clontech, Palo Alto, Calif.) originally developed by Gossen and Bujard (Gossen and Bujard, 1992; Gossen et al., 1995). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli. The tetracycline operator sequence to which the tetracycline repressor binds, and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tertracycline repressor. Thus in the absence of doxycycline, transcription is constitutively on. In the Tet-On™ system, the tetracycline repressor is not wild type and in the presence of doxycycline activates transcription. For gene therapy vector production, the Tet-Off™ system would be preferable so that the producer cells could be grown in the presence of tetracycline or doxycycline and prevent expression of a potentially toxic transgene, but when the vector is introduced to the patient, the gene expression would be constitutively on.

In some circumstances, it may be desirable to regulate expression of a transgene in a gene therapy vector. For example, different viral promoters with varying strengths of activity may be utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoetic cells is desired, retroviral promoters such as the LTRs from MLV or MMTV are often used. Other viral promoters that may be used depending on the desired effect include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. Similarly, the following promoters may be used to target gene expression in other tissues (Table 2).

TABLE 2
Tissue specific promoters

| Tissue | Promoter |
|---|---|
| Pancreas | insulin |
| | elastin |
| | amylase |
| | pdr-1 pdx-1 |
| | glucokinase |
| Liver | albumin PEPCK |
| | HBV enhancer |
| | alpha fetoprotein |
| | apolipoprotein C |
| | alpha-1 antitrypsin |
| | vitellogenin, NF-AB |
| | Transthyretin |
| Skeletal muscle | myosin H chain |
| | muscle creatine kinase |
| | dystrophin |
| | calpain p94 |
| | skeletal alpha-actin |
| | fast troponin 1 |
| Skin | keratin K6 |
| | keratin K1 |
| Lung | CFTR |
| | human cytokeratin 18 (K18) |
| | pulmonary surfactant proteins A, B and C |
| | CC-10 |
| | P1 |
| Smooth muscle | sm22 alpha |
| | SM-alpha-actin |
| Endothelium | endothelin-1 |
| | E-selectin |
| | von Willebrand factor |
| | TIE (Korhonen et al., 1995) |
| | KDR/flk-1 |
| Melanocytes | tyrosinase |
| Adipose tissue | lipoprotein lipase (Zechner et al., 1988) |
| | adipsin (Spiegelman et al., 1989) |
| | acetyl-CoA carboxylase (Pape and Kim, 1989) |
| | glycerophosphate dehydrogenase (Dani et al., 1989) |
| | adipocyte P2 (Hunt et al., 1986) |
| | β-globin |

In certain indications, it may be desirable to activate transcription at specific times after administration of the gene therapy vector. This may be done with such promoters as those that are hormone or cytokine regulatable. For example in gene therapy applications where the indication is a gonadal tissue where specific steroids are produced or routed to, use of androgen or estrogen regulated promoters may be advantageous. Such promoters that are hormone regulatable include MMTV, MT-1, ecdysone and RuBisco. Other hormone regulated promoters such as those responsive to thyroid, pituitary and adrenal hormones are expected to be useful in the present invention. Cytokine and inflammatory protein responsive promoters that could be used include K and T Kininogen (Kageyama et al., 1987), c-fos, TNF-alpha, C-reactive protein (Arcone et al., 1988), haptoglobin (Oliviero et al., 1987), serum amyloid A2, C/EBP alpha, IL-1, IL-6 (Poli and Cortese, 1989), Complement C3 (Wilson et al., 1990), IL-8, alpha-I acid glycoprotein (Prowse and Baumann, 1988), alpha-1 antitypsin, lipoprotein lipase (Zechner et al., 1988), angiotensinogen (Ron et al., 1991), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 antichymotrypsin.

It is envisioned that cell cycle regulatable promoters may be useful in the present invention. For example, in a bi-cistronic gene therapy vector, use of a strong CMV promoter to drive expression of a first gene such as p16 that arrests cells in the G1 phase could be followed by expression of a second gene such as p53 under the control of a promoter that is active in the G1 phase of the cell cycle, thus providing a “second hit” that would push the cell into apoptosis. Other promoters such as those of various cyclins, PCNA, galectin-3, E2F1, p53 and BRCA1 could be used.

Tumor specific promoters such as osteocalcin, hypoxia-responsive element (HRE), MAGE-4, CEA, alpha-fetoprotein, GRP78/BiP and tyrosinase also may be used to regulate gene expression in tumor cells. Other promoters that could be used according to the present invention include Lac-regulatable, chemotherapy inducible (e.g. MDR), and heat (hyperthermia) inducible promoters, Radiation-inducible (e.g., EGR (Joki et al., 1995)), Alpha-inhibin, RNA pol III tRNA met and other amino acid promoters, U1 snRNA (Bartlett et al., 1996), MC-1, PGK, -actin and alpha-globin. Many other promoters that may be useful are listed in Walther and Stein (1996).

It is envisioned that any of the above promoters alone or in combination with another may be useful according to the present invention depending on the action desired. In addition, this list of promoters should not be construed to be exhaustive or limiting, those of skill in the art will know of other promoters that may be used in conjunction with the promoters and methods disclosed herein.

b. Enhancers

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters additional to the tissue specific promoters listed above, cellular promoters/enhancers and inducible promoters/enhancers that could be used in combination with the nucleic acid encoding a gene of interest in an expression construct (Table 3 and Table 4). Additionally, any promoter/enhancer combination (as per the Eukaryotic Promoter Data Base EPDB) could also be used to drive expression of the gene. Eukaryotic cells can support cytoplasmic transcription from certain bacteria promoters if the appropriate bacterial polymerase is provided, either as part of the delivery complex or as an additional genetic expression construct.

TABLE 3
ENHANCER
Immunoglobulin Heavy Chain
Immunoglobulin Light Chain
T-Cell Receptor
HLA DQ α and DQ β
β-Interferon
Interleukin-2
Interleukin-2 Receptor
MHC Class II 5
MHC Class II HLA-DRα
β-Actin
Muscle Creatine Kinase
Prealbumin (Transthyretin)
Elastase I
Metallothionein
Collagenase
Albumin Gene
α-Fetoprotein
τ-Globin
β-Globin
e-fos
c-HA-ras
Insulin
Neural Cell Adhesion Molecule (NCAM)
α1-Antitrypsin
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus
Gibbon Ape Leukemia Virus

TABLE 4

| Element | Inducer |
|---|---|
| MT II | Phorbol Ester (TPA) |
| | Heavy metals |
| MMTV (mouse mammary tumor | Glucocorticoids |
| virus) | |
| β-Interferon | poly(rI)X |
| | poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | FMA |
| Thyroid Stimulating Hormone α | Thyroid Hormone |
| Insulin E Box | Glucose |

In preferred embodiments of the invention, the expression construct comprises a virus or engineered construct derived from a viral genome. The ability of certain viruses to enter cells via receptor-mediated endocytosis and to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

c. Polyadenylation Signals

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human or bovine growth hormone and SV40 polyadenylation signals. Also contemplated as an element of the expression cassette is a terminator. These elements can serve to enhance message levels and to minimize read through from the cassette into other sequences.

7. Methods of Gene Transfer

In order to mediate the effect transgene expression in a cell, it will be necessary to transfer the therapeutic expression constructs of the present invention into a cell. Such transfer may employ viral or non-viral methods of gene transfer. This section provides a discussion of methods and compositions of gene transfer.

I. Viral Vector-Mediated Transfer

The BMPR1A gene is incorporated into an adenoviral infectious particle to mediate gene transfer to a cell. Additional expression constructs encoding other therapeutic agents as described herein also may be transferred via viral transduction using infectious viral particles, for example, by transformation with an adenovirus vector of the present invention as described herein below. Alternatively, retroviral or bovine papilloma virus may be employed, both of which permit permanent transformation of a host cell with a gene(s) of interest. Thus, in one example, viral infection of cells is used in order to deliver therapeutically significant genes to a cell. Typically, the virus simply will be exposed to the appropriate host cell under physiologic conditions, permitting uptake of the virus. Though adenovirus is exemplified, the present methods may be advantageously employed with other viral vectors, as discussed below.

a. Adenovirus

Adenovirus is particularly suitable for use as a gene transfer vector because of its mid-sized DNA genome, ease of manipulation, high titer, wide target-cell range, and high infectivity. The roughly 36 kB viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, 1990). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5′ tripartite leader (TL) sequence which makes them preferred mRNAs for translation.

In order for adenovirus to be optimized for gene therapy, it is necessary to maximize the carrying capacity so that large segments of DNA can be included. It also is very desirable to reduce the toxicity and immunologic reaction associated with certain adenoviral products. The two goals are, to an extent, coterminous in that elimination of adenoviral genes serves both ends. By practice of the present invention, it is possible achieve both these goals while retaining the ability to manipulate the therapeutic constructs with relative ease.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay et al., 1984). Therefore, inclusion of these elements in an adenoviral vector should permit replication.

In addition, the packaging signal for viral encapsidation is localized between 194-385 bp (0.5-1.1 map units) at the left end of the viral genome (Hearing et al., 1987). This signal mimics the protein recognition site in bacteriophage λ DNA where a specific sequence close to the left end, but outside the cohesive end sequence, mediates the binding to proteins that are required for insertion of the DNA into the head structure. El substitution vectors of Ad have demonstrated that a 450 bp (0-1.25 map units) fragment at the left end of the viral genome could direct packaging in 293 cells (Levrero et al., 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element, as provided for in the present invention, derives from the packaging function of adenovirus.

It has been shown that a packaging signal for adenovirus exists in the left end of the conventional adenovirus map (Tibbetts, 1977). Later studies showed that a mutant with a deletion in the E1A (194-358 bp) region of the genome grew poorly even in a cell line that complemented the early (E1A) function (Hearing and Shenk, 1983). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved towards the interior of the Ad5 DNA molecule (Hearing et al, 1987).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals are packaged selectively when compared to the helpers. If the preference is great enough, stocks approaching homogeneity should be achieved.

b. Retrovirus

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed Ψ, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990).

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and Ψ components is constructed (Mann et al., 1983). When a recombinant plasmid containing a human cDNA, together with the retroviral LTR and T sequences is introduced into this cell line (by calcium phosphate precipitation for example), the T sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is collected, optionally concentrated, and used for gene transfer. Retroviral vectors are able to infect a broad variety of cell types. However, integration and stable expression of many types of retroviruses require the division of host cells (Paskind et al., 1975).

An approach designed to allow specific targeting of retrovirus vectors recently was developed based on the chemical modification of a retrovirus by the chemical addition of galactose residues to the viral envelope. This modification could permit the specific infection of cells such as hepatocytes via asialoglycoprotein receptors, should this be desired.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, the infection of a variety of human cells that bore those surface antigens was demonstrated with an ecotropic virus in vitro (Roux et al., 1989).

c. Adeno-Associated Virus

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

The terminal repeats of the AAV vector can be obtained by restriction endonuclease digestion of AAV or a plasmid such as p201, which contains a modified AAV genome (Samulski et al., 1987), or by other methods known to the skilled artisan, including but not limited to chemical or enzymatic synthesis of the terminal repeats based upon the published sequence of AAV. The ordinarily skilled artisan can determine, by well-known methods such as deletion analysis, the minimum sequence or part of the AAV ITRs which is required to allow function, i.e., stable and site-specific integration. The ordinarily skilled artisan also can determine which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, 1996; Chatterjee et al., 1995; Ferrari et al., 1996; Fisher et al., 1996; Flotte et al., 1993; Goodman et al., 1994; Kaplitt et al., 1994; 1996, Kessler et al., 1996; Koeberl et al., 1997; Mizukami et al., 1996).

AAV-mediated efficient gene transfer and expression in the lung has led to clinical trials for the treatment of cystic fibrosis (Carter and Flotte, 1996; Flotte et al., 1993). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor 1× gene delivery to the liver, and potentially of myocardial infarction by vascular endothelial growth factor gene to the heart, appear promising since AAV-mediated transgene expression in these organs has recently been shown to be highly efficient (Fisher et al., 1996; Flotte et al., 1993; Kaplitt et al., 1994; 1996; Koeberl et al., 1997; McCown et al., 1996; Ping et al., 1996; Xiao et al., 1996).

d. Other Viral Vectors

Other viral vectors may be employed as expression constructs in the present invention. Vectors derived from viruses such as vaccinia virus (Ridgeway, 1988; Baichwal and Sugden, 1986; Coupar et al., 1988) canary pox virus, and herpes viruses may be employed. These viruses offer several features for use in gene transfer into various mammalian cells.

II. Non-Viral Transfer

DNA constructs of the present invention are generally delivered to a cell, in certain situations, the nucleic acid to be transferred is non-infectious, and can be transferred using non-viral methods.

Several non-viral methods for the transfer of expression constructs into cultured mammalian cells are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988).

Once the construct has been delivered into the cell the nucleic acid encoding the therapeutic gene may be positioned and expressed at different sites. In certain embodiments, the nucleic acid encoding the therapeutic gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In a particular embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The addition of DNA to cationic liposomes causes a topological transition from liposomes to optically birefringent liquid-crystalline condensed globules (Radler et al., 1997). These DNA-lipid complexes are potential non-viral vectors for use in gene therapy.

Liposome-mediated nucleic acid delivery and expression of foreign DNA in vitro has been very successful. Using the β-lactamase gene, Wong et al., (1980) demonstrated the feasibility of liposome-mediated delivery and expression of foreign DNA in cultured chick embryo, HeLa, and hepatoma cells. Nicolau et al., (1987) accomplished successful liposome-mediated gene transfer in rats after intravenous injection. Also included are various commercial approaches involving “lipofection” technology.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear nonhistone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems which can be employed to deliver a nucleic acid encoding a therapeutic gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferring (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also may be specifically delivered into a cell type such as prostate, epithelial or tumor cells, by any number of receptor-ligand systems with or without liposomes. For example, the human prostate-specific antigen (Watt et al., 1986) may be used as the receptor for mediated delivery of a nucleic acid in prostate tissue.

In another embodiment of the invention, the expression construct may simply consist of naked recombinant DNA or plasmids. Transfer of the construct may be performed by any of the methods mentioned above which physically or chemically permeabilize the cell membrane. This is applicable particularly for transfer in vitro, however, it may be applied for in vivo use as well. Dubensky et al., (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding a CAM also may be transferred in a similar manner in vivo and express CAM.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads

8. Formulations and Routes for Administration to Patients

Where clinical applications are contemplated, it will be necessary to prepare pharmaceutical compositions—expression vectors, virus stocks, proteins, antibodies and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral and rectal. Alternatively, administration may be by intradermal, subcutaneous, intramuscular, intraperitoneal or intravenous injection. Such compositions would normally be administered as pharmaceutically acceptable compositions, described supra.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient also may be dispersed in gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

9. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Materials and Methods

Patients and Families. The inventor obtained blood samples from kindred members after obtaining informed consent, reviewed medical records (including pathology, endoscopy and surgical reports) to confirm the diagnosis of JP, and reviewed pathology slides, where available. Individuals were classified as “affected” if they had histologic evidence of upper gastrointestinal or colorectal juvenile polyps, and as ‘unknown’ if there was no definitive histologic diagnosis of juvenile polyps. Control patient samples were selected at random from blood samples obtained from anonymous donors at the University of Iowa outpatient laboratory.

Genotyping. DNA was extracted from whole-blood samples using a salting-out procedure (Miller et al., 1988), and the genome screen were performed using the Weber screening set 8a simple tandem repeat polymorphisms markers (Research Genetics), as described previously (Howe et al., 1998a).

Linkage Analysis and Mapping. Two-point linkage calculations were performed assuming autosomal dominant inheritance, a gene frequency of 1 in 100,000, and 95% penetrance (Howe et al., 1998a), using the MLINK subroutine of the FASTLINK 2.3 version of the LINKAGE program package (Cottingham et al., 1993). Haplotypes were constructed manually for each family assuming the least possible number of recombination events. Markers were ordered according to the Whitehead Human Physical Mapping project http://carbon.wi.mit.edu:8000/cgi-bin/contig/phys_map) and the Center for Medical Genetics maps (http://research.marshfieldclinic.org/genetics). The GeneBridge 4 radiation hybrid DNA panel (Research Genetics) and the WICGR mapping service (http://carbon.wi.mit.edu:8000/cgi-bin/contig/rhmapper.pl) were used to map BMPR1A exon 1 and D10S573.

Generation of Simple Tandem Repeat Polymorphisms from BMPR1A Region. A basic local alignment search tool search (http://www.ncbi.nlm.nih.gov/BLAST), using the BMPR1A cDNA sequence 8, was performed which showed homology to the 173,675-bp BAC clone RP11-420K10. The BAC sequence was searched for simple tandem repeat sequences by alignment of 20-bp sequences of di-, tri- and tetranucleotide repeat elements. The inventor then selected the following primer pairs flanking these repeat elements using the Primer3 program (http://www-genome.wi.mit.edu/cgi-bin/primer/primer3_www.cgi):

ALK3CA-1a (5-GATCCAGAAACCAAGGGAAA-3) and

ALK3CA-1b (5-TGGTAGATGGAGGTGGGGTA-3) (186-bp product);

ALK3GGAA-1a (5-CACACTGCAGGTGCTCTACAA-3) and

ALK3GGAA-1b (5-CTTGGGCAACAGAGCAAGAT-3) (210-bp product).

These primers were used to amplify DNA from 100 control patients, then separated the products by electrophoresis through 6% denaturing polyacrylamide gels and silver-stained the gels. The allele frequencies and heterozygosity were determined for each marker, and then used each for genotyping in JP families.

Definition of BMPR1A Intron-Exon Boundaries. BMPR1A intron-exon boundaries were defined by alignment of short segments of the BMPR1A cDNA sequence to the RP11-420K10 BAC sequence using the Sequencher Program (Gene Codes Corporation, v.3.0.1). After defining the intron-exon boundaries, the following primers were selected from the introns flanking each exon using the Primer3 program: exon1, ALK3-1a (5-TGTCAAGTGCTTGCGATCTT-3) and ALK3-1b (5-GGCTGGGCCTAACTATTCAA-3) (289-bp product); exon 2, ALK3-2a (5-TTGTCACGAAACAATGAGCTTT-3) and ALK3-2b (5-AACTCTTAAGAAGGGCTGCAT-3) (257-bp product); exon 3, ALK3-3a (5-AGGCCATCTGTACCTGTTCAC-3) and ALK3-3b (5-ATATGGCCCCTCCCTTCTTT-3) (246-bp product); exons 4 and 5, ALK3-4/5a (5-TAAAATTTGCAGGCCCCTTT-3) and ALK3-4/5b (5-GCTTTACAAACAGCGGTTGA-3) (519-bp products; exon 6, ALK3-6a (5-GGATTCTTTCTGAGGGAAGGA-3) and ALK3-6b (5-TCCACCATCATGAGGACACA-3) (317-bp product); exon 7, ALK3-7a: 5-CCCTTTGCCAGTCTTAATGG-3, ALK3-7b: 5-AGGCTTCCACCTGTACCTCA-3 (323-bp product); exon 8, ALK3-8a (5-TGAGCATTACTTCTCCCTAGCC-3) and ALK3-8b (5-TTCAAAACAGTGGGGCAAAG-3) (394-bp product); exon 9, ALK3-9a (5-CAACTTGGACCTTGGCTTTC-3) and ALK3-9b (5-CATGGCATGCCTGTATCAAA-3) (361-bp product); exons 10 and 11, ALK3-10/11a (5-AACCATTTTTGTGCCCATGT-3) and ALK3-10/11b (5-CACTCTAATTCCACCCATGC-3) (456-bp product). PCR conditions were optimized for each primer pair using control DNA samples.

DNA Sequencing and Mutation Analysis. Amplified DNA was obtained from kindred members using BMPR1A primers in a 30-μl reaction volume, then separated products by electrophoresis through 2% agarose gels. Gel-purified PCR products (QIAquick, Qiagen) were sequenced in both directions with dye terminators (Applied Biosystems Prism Cycle Sequencing), using the PCR primers as sequencing primers, and determined their sequences using a Model 373 automated sequencer and ABI analysis software (Applied Biosystems). Additional family members were tested for mutations by sequencing, SSCP and/or denaturing gel electrophoresis of specific exons. A four-bp deletion in exon 1 in the D kindred was resolved by electrophoresis through 6% denaturing polyacrylamide gels and silver-stained the gels. The inventor evaluated the mutation in exon 7 in the E kindred by SSCP using the primers 5-CAGCGAACTATTGCCAAACA-3 and 5-ATGGCGCATTAGCACAGTTT-3 (177-bp product); for the B kindred, the SSCP primers were 5-AAGTATGGATGGGCAAATGG-3 and 5-ATGGCGCATTAGCACAGTTT-3 (116-bp product). To examine the one-bp deletion in exon 8 in the S kindred, family members were amplified using the primers 5-CAGGTTCCTGGACTCAGCTC-3 and 5-CTTTCCTTGGGTGCCATAAA-3 (170-bp product), then separated the products by electrophoresis through 10% denaturing polyacrylamide gels and identified them by silver-staining. The exon 1 polymorphism was examined by amplifying DNA from each individual using the ALK3-1a and ALK3-1b primers, then digested the products with Hinf1. These products were separated by electrophoresis through 6% polyacrylamide gels and identified them by silver staining. For mutational analysis of sporadic cancers, the PCR products (Qiagen) were gel-purified and analyzed the sequencing reactions on an SCE-9610 96-well capillary electrophoresis system (SpecrtruMedix Corporation).

Laser Capture Microdissection. Paraffin-embedded tissue blocks containing juvenile polyp tissue from JP family members were cut into sections 5 ∝m in thickness and stained slides with hematoxylin and eosin. A Pixcell II image archiving workstation (Arcturus Engineering) was used to obtain separate laser captures of lamina propria and epithelial cells using an amplitude of 50 mW, a duration of 800 μis and a 7.5-μm beam. DNA was extracted from the Capsure lids (Arcturus Engineering) containing microdissected tissue in 50 μl lysis buffer (10 mM Tris, pH 8.0, 1 mM EDTA, 1% Tween 20 and 0.1 mg/ml proteinase K) incubated overnight at 37° C.

LOH Studies. For sporadic colorectal cancers, the markers ALK3GGAA, CHLC.GATA81F06 and D10S1242 were amplified in a10-μl volume using 4 ng genomic DNA and DNA extracted from cancer xenografts or cell lines, as described (Thiagalingam et al., 2001). Five μl of a 1:20 dilution of each PCR product was diluted with 45 μl Hi-Di formamide (Applied Biosystems), then analyzed each sample using an SCE-9610 capillary electrophoresis system (SpectruMedix Corporation). For juvenile polyps, 5-10 ng genomic DNA and DNA were extracted by laser capture microdissection from lamina propria and epithelium to amplify D10S573, ALK3CA, ALK3GGAA, the mutated exon of electrophoresis through 6% polyacrylamide gels and identified them by silver staining.

GenBank Accession Numbers. BMPR1A cDNA, NM004329; BAC clone RP11-420K10, AC021036; ALK3CA, AF364128; ALK3GGAA, AF364128.

### EXAMPLE 2

### Results and Discussion

The inventor chose four families with multiple affected members, but without mutations in MADH4 or PTEN for linkage analyses, and found lod scores >1 with the markers D10S2327, GATA115E01 and D10S677 from chromosome 10q. He then genotyped additional markers from this region and calculated a maximum lod score of 2.33 at θ=0.10 with D10S573 (Table 5). This region corresponded to chromosome 10q22−23 and was near both PTEN (Nelen et al., 1996; Liaw et al., 1997) and a putative JP locus called JP1 (Jacoby et al., 1997b). Because mutations in PTEN had already been excluded in these kindreds by direct sequencing, the inventor searched the Unigene and LocusLink databases from the National Center for Biotechnology Information server for other genes mapping to 10q22-23. One of these genes was BMPR1A (also known as ALK-3), which mediates bone morphogenic protein intracellular signaling through MADH4 (Kretzschmar et a., 1997; Henningfeld et al., 2000). Given the association between MADH4 and JP in other kindreds, this gene was analyzed in detail. Two new microsatellite markers from a bacterial artificial chromosome (BAC) clone containing BMPR1A were developed. One of these was a GGAA repeat (ALK3GGAA), located 76.3 kb upstream of BMPR1A exon 1, with eight alleles and a heterozygosity of 72%. The other was a CA repeat (ALK3CA) 49.4 kb upstream of BMPR1A exon 1, with 12 alleles and a heterozygosity of 68%. Maximum lod scores of 4.74 and 4.17 at θ=0.00 with ALK3CA and ALK3GGAA, were observed, respectively, by linkage analysis in these JP families. BMPR1A 3.1 cR was placed telomeric to WI-5226 and 7.9 cR centromeric to AFM225YD12 (with D10S1242 lying just centromeric to AFM225YD12) by radiation-hybrid mapping, and placed D10S573 3.5 cR telomeric to 10S1427 and 2.9 cR centromeric to WI-5226. Using haplotype analysis for the markers listed in Table 5, seven affected individuals were identified with essential recombination events, further refining the JP locus to between D10S573 and D10S!242. No recombinants with ALK3CA and ALK3GGAA were found in these four families.

TABLE 5
Chromosome 10q22-23 Markers in Four Juvenile Polyposis
Kindreds (LOD Scores)

| | | Posi- | | | | | |
|---|---|---|---|---|---|---|---|
| | Position | tion | | Z at θ = | | | θ at |
| Marker | (cM)¹ | (cR)² | 0.00 | 0.05 | 0.10 | Zmax | Zmax |
| D1081432 | 93.92 | — | <−2 | −1.32 | −0.47 | 0.16 | 0.25 |
| D10S2327 | 100.92 | — | <−2 | 1.20 | 1.32 | 1.32 | 0.10 |
| D10S573 | 106.11 | 490³ | −1.01 | 2.17 | 2.33 | 2.33 | 0.10 |
| ALK3CA | — | 496⁴ | 4.74 | 4.21 | 3.68 | 4.74 | 0.00 |
| ALK3GGAA | — | — | 4.17 | 3.75 | 3.31 | 4.17 | 0.00 |
| D10S1242 | 109.98- | 504 | <−2 | 0.28 | 0.93 | 1.16 | 0.18 |
| | 112.58⁵ | | | | | | |
| GATA115E01 | 112.58 | — | <−2 | 0.35 | 0.88 | 1.03 | 0.16 |
| D10S677 | 117.42 | 522-5 | <−2 | 0.46 | 1.17 | 1.41 | 0.17 |
| D10S1239 | 125.4 | — | <−2 | −1.04 | −0.15 | 0.47 | 0.26 |

¹Based on sex-averaged Center for Medical Genetics map.
²Based on Whitehead Human Physical Mapping Project.
³Based on Genebridge (ref. 4) panel, mapping 3.46 cR telomeric to D10S1427, which is at 487 cR on the Whitehead physical map.
⁴Based upon results using exon 1 of BMPR1A with the Genebridge4 panel, which mapped 3.1 cR telomeric to WI-5226 (493 cR on Whitehead physical map) and 7.9 cR centromeric to AFM225YD12 (504 cR on Whitehead physical map).
⁵Maps between AFMb362YG5 and AFM287ZE1 on Whitehead physical map, with these markers at 109.98 and 112.58 cM, respectively, on Center for Medical Genetics map.

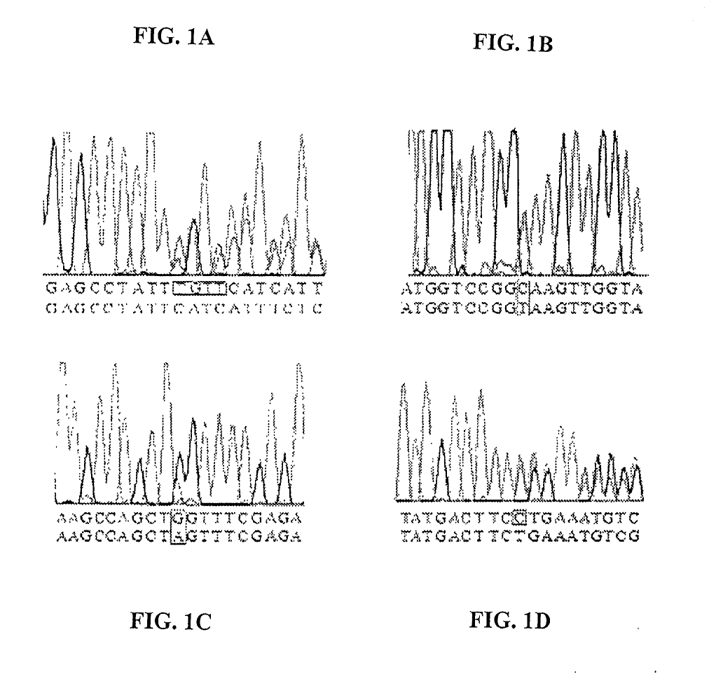

BMPR1A consists of 11 exons distributed over 52,157 bp. The inventor determined the complete sequence of each exon of BMPR1A in selected members of each kindred. In the D kindred, a four-bp deletion was detected in exon 1 (44-47delTGTT), resulting in a stop codon at nucleotides 104-106 (Table 6). In the E kindred, there was a transition at nucleotide 715, changing codon 239 from a glutamine to a stop codon (Q239X). In the B kindred, there was a transition at nucleotide 812, changing a tryptophan to a stop codon (W271X). In the S kindred, there was a one-bp deletion in exon 8 (961delC), creating a stop at the next codon. The sequences corresponding to each mutation are shown in FIGS. 1A-1D.

TABLE 6
Germline Mutations in BMPR1A Found in Four Unrelated JP Kindreds

| | | | Nucleotide | Predicted | Control |
|---|---|---|---|---|---|
| Kindred | Codon | (Exon) | Change | Effect | Individuals |
| D | 15-16 | (1) | 44-47delTGTT | Frameshift, | 0/250 |
| | | | | stop at | |
| | | | | codon 35-36 | |
| E | 239 | (7) | 715C > T | Q239X | 0/250 |
| B | 271 | (7) | 812G > A | W271X | 0/250 |
| S | 321 | (8) | 961delC | Frameshift, | 0/250 |
| | | | | stop | |
| | | | | at codon 321 | |

Six additional mutations from other kindreds include 245G→A (C82Y) (exon 3), 349C→T (Q117X) (exon 4), 262G→T (E84X) (exon 3), 353delT (stop 122-123) (exon 4), 1061delG (stop 363-364) (exon 8), 184T→G (Y62D) (exon 2), 674delT (stop 259-260) (exon 6), and 1013C→T (A338D) (exon 8) (data not shown).

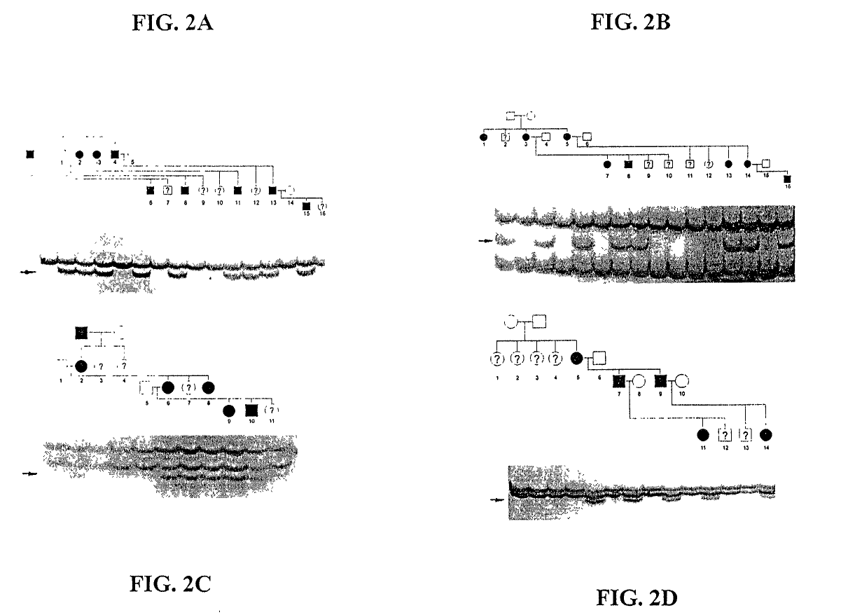

All eight affected members of the D kindred had the four-bp deletion, as did one individual at risk (43 years of age). The inventors did not identify this mutation in four other family members at risk without a diagnosis of JP (5-51 years of age; FIG. 2A). All eight affected members of the E kindred had the substitution in exon 7, whereas none of five family members (41-71 years of age) with-out JP had the mutation (FIG. 2B). In the B kindred, all five affected family members had the substitution in exon 7, as did one (35 years of age) of four family members without a diagnosis of JP (8-70 years of age; FIG. 2C). In the S kindred, all five affected kindred members had the deletion in exon 8, and this mutation was not found in any of the six family members without JP (19-77 years of age; FIG. 2D). The inventor did not find any of the four mutations described here in 250 normal control individuals (Table 6). A polymorphism was identified at nucleotide 4 (4A→C), and in 109 unrelated individuals, allele frequencies were 12% for the A/A genotype, 35% for A/C and 53% for C/C. Accordingly, this encoded for threonine at amino acid 2 (ACT, as in the published cDNA sequence) (ten Dijke et al., 1993) in 29% of chromosomes and for proline (CCT) in 71%.

Historically, TGF-β was thought to be the principal member of the TGF-β superfamily controlling the growth of colonic epithelial cells. However, some data have supported the idea that other, unspecified members of this family may be more important than TGF-β (Dai et al., 1999; Sirard et al., 2000; Fink et al., 2001). Although the involvement of BMP has not been indicated before in colorectal tumorigenesis, BMP receptors are widely distributed and are not confined to bone (Iwasaki et al., 1995). Moreover, there is evidence that BMPs can negatively regulate neoplastic growth (Raida et al., 1999; Kleeff et al., 1999). To determine whether BMPR1A alterations occur in sporadic cancers, the inventor evaluated 139 sporadic colorectal cancers for LOH at 10q22-23 using three simple tandem repeat polymorphisms spanning the BMPR1A region (CHLC.GATA81F06, ALK3GGAA and D10S1242). LOH was detected in 34 of 139 (24%) tumors analyzed. Genomic sequencing of all 11 BMPR1A exons and intron-exon boundaries in 22 tumors showing LOH was performed. No somatic mutations were identified in the remaining alleles of these tumors, challenging the idea of the involvement of this gene in colorectal neoplasia unassociated with JP.

The nonsense mutations reported in these four JP kindreds encode BMP receptors that lack the intracellular serine-threonine kinase domain (ten Dijke et al., 1993), and are predicted to result in loss of BMP-mediated intracellular signaling. The finding that germline mutations in both MADH4 and BMPR1A result in the JP phenotype raises the question of whether the effects of MADH4 are mediated through alterations in BMP signaling rather than through other TGF-β family members. In addition to their consequences for the diagnosis and management of families with JP, these results provide the first genetic evidence that BMPs are important to the control of epithelial neoplasia.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference:

“Manipulating the Mouse Embryo; A Laboratory Manual” 2nd edition eds., Hogan, Beddington, Costantimi and Long, Cold Spring Harbor Laboratory Press, 1994;

“Remington's Pharmaceutical Sciences” 15th Edition, pages 1035-1038 and 1570-1580

Arap et al., Cancer Res., 55:1351-1354, 1995.

Arcone et al., Nucl. Acids Res., 16(8): 3195-3207, 1988.

Jarvinen & Franssila, Gut 25, 792-800, 1984.

Baichwal & Sugden, In: Gene Transfer, Kucherlapati R, ed., New York, Plenum Press, pp. 117-148, 1986.

Baptist & Sabatini, Hum. Pathol. 16:1061-1063, 1985.

Bartlett et al., Proc. Nat'l Acad. Sci. USA, 93:8852-8857, 1996.

Bassam et al., Analytical Biochemistry 196, 80-83, 1991.

Bedzyk et al., J. Biol. Chem., 265:18615, 1990

Bellus, J. Macromol. Sci. Pure Appl. Chem, A311: 1355-1376, 1994.

Bentley et al., Am. J. Gastroenterol. 84:1456-1459, 1989.

Benvenisty & Neshif, Proc. Nat'l Acad. Sci. USA, 83:9551-9555, 1986.

Brack-Werner et al., Genomics 4:68-75, 1989.

Brinster et al., Proc. Nat'l Acad. Sci. USA, 82: 4438-4442, 1985.

Burt et al., Bulletin of the World Health Organization 68, 655-664, 1993.

Bussemakers et al., Cancer Res., 52:2916-2922, 1992.

Caldas et al., Nat'l Genet., 8:27-32, 1994.

Carle & Olson Nucleic Acids Research 12, 5647-5664, 1984.

Carter & Flotte, Ann. N.Y. Acad. Sci., 770:79-90, 1995.

Carter et al., Proc. Nat'l Acad. Sci. USA, 87:8751-8755, 1990.

Chatterjee, et al., Ann. N.Y. Acad. Sci., 770:79-90, 1995.

Chaudhary et al., Proc. Nat'l Acad. Sci., 87:9491, 1990

Chen & Faller, J. Biol. Chem., 271:2376, 1996.

Chen & Okayama, Mol. Cell Biol., 7:2745-2752, 1987.

Cheng et al., Cancer Res., 54:5547-5551, 1994.

Cheng et al., Nature, 379:554, 1996.

Cho et al., Genomics 19:525-531, 1994.

Coffin, In: Virology, ed., New York: Raven Press, pp. 1437-1500, 1990.

Cottingham et al., Faster sequential linkage computations. Am. J. Hum. Genet. 53, 252-263, 1993.

Coupar et al., Gene, 68:1-10, 1988

Culver et al., Science, 256:1550-1552, 1992.

Dai et al., G1 cell cycle arrest and apoptosis induction by nuclear Smad4/Dpc4: phenotypes reversed by a tumorigenic mutation. Proc. Nat'l Acad. Sci. USA 96, 1427-1432, 1999.

Dani, et al., J. Biol. Chem., 264:10119-10125, 1989.

Davey et al., EPO No. 329 822.

Devereux et al., Carcinogenesis; 18(9): 1751-1755. 1997

Dubensky et al., Proc. Nat'l Acad. Sci. USA, 81:7529-7533, 1984.

Edelman & Crossin, Annu. Rev. Biochem., 60:155-190, 1991.

Eng & Ji, Molecular classification of the inherited hamartoma polyposis syndromes: Clearing the muddied waters. Am. J. Hum. Genet. 62, 1020-1022, 1998.

EPO No. 320 308,

Eppert et al., Cell 86, 543-552, 1996.

Fazeli et al., Nature 386, 796-804, 1997.

Fearon & Vogelstein, Cell, 61:759-767, 1990.

Fearon et al., Science 247, 49-247, 1990.

Fechheimer et al., Proc. Nat'l Acad. Sci. USA, 84:8463-8467, 1987.

Feinberg & Vogelstein, Analytical Biochemistry 137, 266-267, 1984.

Ferkol et al., FASEB J., 7:1081-1091, 1993.

Ferrari et al., J. Virol., 70:3227-3234, 1996.

Fink et al., Transforming growth factor-beta-induced growth inhibition in a smad4 mutant colon adenoma cell line. Cancer Res. 61, 256-260, 2001.

Fisher et al., J. Virol., 70:520-532, 1996.

Flotte et al., Proc. Nat'l Acad. Sci. USA, 90:10613-10617, 1993.

Fodor et al., Science, 251:767-773, 1991.

Fraley et al., Proc. Nat'l Acad. Sci. USA, 76:3348-3352, 1979.

Freifelder, Physical Biochemistry Applications to Biochemistry and Molecular Biology, 2nd ed. Wm. Freeman and Co., New York, N.Y., 1982.

Frixen et al., J. Cell Biol., 113:173-185, 1991.

Frohman, In: PCR Protocols: A Guide To Methods And Applications, Academic Press, N.Y., 1990.

GB Application 2 202 328

Gentry et al., Multiple hamartoma syndrome (Cowden disease). Arch. Dermatol. 109, 521-525, 1974.

Ghosh-Choudhury et al., EMBO J., 6:1733-1739, 1987.

Ghosh and Bachhawat, In: Liver Diseases, Targeted Diagnosis and Therapy Using Specific Receptors and Ligands. Wu et al., eds., Marcel Dekker, New York, pp. 87-104, 1991.

Gingeras et al., PCT Application WO 88/10315,

Goodman et al., Blood, 84:1492-1500, 1994.

Goodman et al., Cancer 43, 1906-1913, 1979.

Gopal, Mol. Cell Biol., 5:1188-1190, 1985.

Gossen and Bujard, Proc. Nat'l Acad. Sci. USA, 89:5547-5551, 1992.

Gossen et al., Science, 268:1766-1769, 1995.

Grady et al., Cancer Res., 59, 320-324, 1999.

Graham & Prevec, In: Methods in Molecular Biology: Gene Transfer and Expression Protocol, E. J. Murray, ed., Humana Press, Clifton, N.J., 7:109-128, 1991.

Graham & van der Eb, Virology, 52:456-467, 1973.

Grau et al., Cancer Res., 57:3929, 1997.

Grotsky et al., Gastroenterology 82:494-501, 1982.

Hacia et al., Nature Genetics, 14:441-447, 1996.

Hahn et al., Cancer Res; 58(6):1124-1126 1998

Hahn et al., Cancer Research 56, 490-494, 1996b.

Hahn et al., Science 271, 350-353, 1996a.

Harland & Weintraub, J. Cell Biol., 101:1094-1099, 1985.

Hay et al., J. Mol. Biol., 175:493-510, 1984.

Hearing & Shenk, J. Mol. Biol. 167:809-822, 1983.

Hearing et al., J. Virol., 67:2555-2558, 1987.

Heldin et al., TGF-αsignaling from cell membrane to nucleus through SMAD proteins. Nature 390, 465-471, 1997.

Hemminki et al., Nat. Genet. 15:87-90, 1997.

Hemminki et al., Nature 391:184-187, 1998.

Henningfeld et al., Smad1 and Smad4 are components of the bone morphogenetic protein-4 (BMP-4)-induced transcription complex of the Xvent-2B promoter. J. Biol. Chem. 275, 21827-21835, 2000.

Hollstein et al., Science, 253:49-53, 1991.

Hoodless et al., MADR1, a MAD-related protein that functions in BMP2 signaling pathways. Cell 85, 489-500, 1996.

Houlston et al., Mutations in DPC4 (SMAD4) cause juvenile polyposis syndrome, but only account for a minority of cases. Hum. Mol. Genet. 7, 1907-1912, 1998.

Howe & Conlon, Surgical Oncology 6, 1-18, 1997.

Howe & Guillem, Surgical Clinics of North America 77, 175-195, 1997.

Howe et al., American Journal of Human Genetics 51, 1430-1442, 1992a.

Howe et al., Clinical Cancer Research 3, 129-133, 1997.

Howe et al., Histology and Histopathology 12, 595-601, 1997.

Howe et al., Human Genetics 91, 199-204, 1993.

Howe et al., Nucleic Acids Research 19, 2518, 1991.

Howe et al., Nucleic Acids Research 20, 1168, 1992.

Howe et al., Science 280, 1086-1088, 1998b.

Howe et al., Am. J. Hum. Genet. 62, 1129-36 1998a.

Howe et al., Surgery 112, 219-226, 1992b.

Howe et al., Surgical Forum 41, 447-450, 1990.

Howe et al., Histol. Histopathol. 12:595, 1997.

Howe et al., The risk of gastrointestinal carcinoma in familial juvenile polyposis. Ann. Surg. Onc. 5, 751-756, 1998.

Hudson et al., Science 270, 1945-1954, 1995.

Hunt et al., Proc. Nat'l Acad. Sci. USA, 83:3786-3790, 1986.

Hursh, et al., Development, 117:1211, 1993 Hussussian et al., Nature Genetics, 15-21, 1994.

Innis et al., PCR Protocols, Academic Press, Inc., San Diego Calif., 1990.

Iwasaki et al., Distribution and characterization of specific cellular binding proteins for bone morphogenetic protein-2. J. Biol. Chem. 270, 5476-5482, 1995.

Jacoby et al., American Journal of Human Genetics 70, 361-364, 1997a.

Jacoby et al., A juvenile polyposis tumor suppressor locus at 10q22 is deleted from nonepithelial cells in the lamina propria. Gastroenterology 112, 1398-1403, 1997b.

Jarvinen, Problems in General Surgery 10, 749-757, 1993.

Jarvinen & Franssila, Familial juvenile polyposis coli: Increased risk of colorectal cancer. Gut 25, 792-800, 1984.

Jass et al., Histopathology 13:619-630, 1988.

Jass, In: Utsunomiya J, Lynch HT (eds) “Hereditary colorectal cancer.” Springer, Tokyo, pp 343-350, 1990.

Jenne et al., Nature Genetics 18, 38-43, 1998.

Joki et al., Human Gene Ther., 6:1507-1513, 1995.

Jones et al., Arch. Pathol. Lab. Med. 111:200-201, 1987.

Kageyama et al., J. Biol. Chem., 262(5):2345-2351, 1987.

Kamb et al., Nature Genetics, 8:22-26, 1994.

Kamb et al., Science, 264:436-440, 1984.

Kaneda et al., Science, 243:375-378, 1989.

Kaplitt et al., Arm. Thor. Surg., 62:1669-1676, 1996.

Kaplitt et al., Nat'l Genet., 8:148-153, 1994.

Kato et al., J. Biol. Chem., 266:3361-3364, 1991.

Keino-Masu et al., Cell 87, 175-185, 1996.

Kessler et al., Proc. Nat'l Acad. Sci. USA, 93:14082-14087, 1996.

Kim et al., Cancer Res., 56:2519, 1996.

Kleeff et al., Bone morphogenetic protein 2 exerts diverse effects on cell growth in vitro and is expressed in human pancreatic cancer in vivo. Gastroenterology 116, 1202-1216, 1999.

Klein et al., Nature, 327:70-73, 1987.

Knudson, et al., Proc. Nat'l Acad. Sci. U.S.A. 72, 5116-5120, 1975.

Knudsonn et al., Science, 270:96, 1995.

Koeberl et al., Proc. Nat'l Acad. Sci. USA, 94:1426-1431, 1997.

Korhonen, et al., Blood, 86(5):1828-1835, 1995.

Kretzschmar et al., The TGF-β family mediator Smad1 is phosphorylated directly and activated functionally by the BMP receptor kinase. Genes Dev. 11, 984-995, 1997.

Kwoh et al., Proc. Nat'l Acad. Sci. USA, 86: 1173, 1989.

Lagna et al., Partnership between DPC4 and SMAD proteins in TGF-β signalling pathways. Nature 383, 832-836, 1996.

Landis et al., CA-A Cancer Journal for Clinicians 48, 6-30, 1998.

Lathrop et al., Am. J. Hum. Gene.t 37:482-498, 1985.

Lathrop, et al., Proc. Nat'l Acad. Sci. USA, 81:3443-3446, 1984.

Leggett et al., Gastroenterology 105, 1313-1316, 1993.

Levrero et al., Gene, 101:195-202, 1991.

Li & Sun, Cancer Res., 57:2124-2129, 1997.

Liaw et al., Germline mutations of the PTEN gene in Cowden disease, an inherited breast and thyroid cancer syndrome. Nature Genet. 16, 64-67, 1997.

Liu et al., Chin. Med. J. (Engl) 4:434-439, 1978.

Liu et al., A human Mad protein acting as a BMP-regulated transcriptional activator. Nature 381, 620-623, 1996.

Lynch et al., Cancer 56, 939-951, 1985.

Lynch et al., Inherited mutations in PTEN that are associated with breast cancer, Cowden disease, and juvenile polyposis. Am. J. Hum. Genet. 61, 12541260, 1997.

Macejak & Sarnow, Nature, 353:90-94, 1991.

MacGorgan et al., Oncogene; 15(9): 1111-1114, 1997

Mann et al., Cell, 33:153-159, 1983.

Marsh et al., Cancer Research 57, 5017-5021, 1997.

Marsh et al., Nature Genetics, 16:333, 1997.

Massague, J., TGF signaling: Receptors, transducers, and Mad proteins. Cell 85, 947-950, 1996.

Matsura et al., Brit. J. Cancer, 66:1122-1130, 1992.

McCown et al., Brain Res., 713:99-107, 1996.

Mehenni et al., Am. J. Hum. Genet. 61:1327-1334,1997.

Miller et al., A simple salting out procedure for extracting DNA from nucleated cells. Nucleic Acids Res. 16, 1215, 1988.

Miller et al., PCT Application WO 89/06700

Mizukami et al., Virology, 217:124-130, 1996.

Mori et al., Cancer Res., 54:3396-3397, 1994.

Morson, Dis. Colon. Rectum. 5:337-344 1962.

Moskaluk et al., Diagnostic Molecular Pathology 6, 85-90, 1997.

Myers, EPO 0273085

Nakamura et al., In: Handbook of Experimental Immunology (4th Ed.), Weir, E., Herzenberg, L. A., Blackwell, C., Herzenberg, L. (eds). Vol. 1, Chapter 27, Blackwell Scientific Publ., Oxford, 1987.

Nelen et al., Localization of the gene for Cowden disease to chromosome 10q22-23. Nature Genet. 13, 114-116, 1996.

Newton et al., Statistical Medicine 13, 839-858, 1994.

Nichols et al., Computer Applications in the Biosciences 9, 757-759, 1993.

Nicolas and Rubenstein, In: Vectors: A survey of molecular cloning vectors and their uses, Rodriguez and Denhardt (eds.), Stoneham: Butterworth, pp. 493-513, 1988.

Nicolau & Sene, Biochim. Biophys. Acta, 721:185-190, 1982.

Nicolau et al., Methods Enzymol., 149:157-176, 1987.

Nishizuka et al., Jpn. J. Cancer Res.; 88(4): 335-339, 1997

Nobori et al., Nature, 368:753-756, 1995.

Ohara et al., Proc. Nat'l Acad. Sci. USA, 86: 5673-5677, 1989.

Okamoto et al., Proc. Nat'l Acad. Sci. USA, 91:11045-11049, 1994.

Olivierio et al., EMBO J., 6(7):1905-1912, 1987.

Olschwang et al., PTEN germline mutations in juvenile polyposis coli. Nature Genet. 18, 12-14, 1998.

Orlow et al., Cancer Res., 54:2848-2851, 1994.

Pape & Kim, Mol. Cell. Biol., 974-982, 1989.

Paskind et al., Virology, 67:242-248, 1975.

PCT/US87/00880

PCT/US89/01025

Pease et al., Proc. Nat'l Acad. Sci. USA, 91:5022-5026, 1994.

Pelletier & Sonenberg, Nature, 334:320-325, 1988.

Perales et al., Proc. Nat'l Acad. Sci. 91:4086-4090, 1994.

Pignon et al., Hum. Mutat., 3: 126-132, 1994.

Ping et al., Microcirculation, 3:225-228, 1996.

Poli & Cortese, Proc. Nat'l Acad. Sci. USA, 86:8202-8206, 1989.

Potter et al., Proc. Nat'l Acad. Sci. USA, 81:7161-7165, 1984.

Prowse & Baumann, Mol Cell Biol, 8(1):42-51, 1988.

Radler et al., Science, 275:810-814, 1997.

Raida et al., Expression, regulation and clinical significance of bone morphogenetic protein 6 in esophageal squamous-cell carcinoma. Int. J. Cancer 83, 38-44, 1999.

Ramaswamy et al., Diseases of the Colon and Rectum 27, 393-398, 1984.

Reiss et al., Cell Growth Differ; 8(4): 407-415, 1997.

Remington's Pharmaceutical Sciences, 15th ed., pp. 1035-1038 and 1570-1580.

Renan, Radiother. Oncol., 19:197-218, 1990.

Ridgeway, In: Vectors: A survey of molecular cloning vectors and their uses, Rodriguez RL, Denhardt DT, ed., Stoneham:Butterworth, pp. 467-492, 1988.

Riggins et al., “Normal PTEN gene in juvenile polyposis.” NOGO 1:1 (//pathology.jhu.edu/nogo/), 1997.

Rippe et al., Mol. Cell Biol., 10:689-695, 1990.

Risinger & Boyd, Hum. Mol. Genet. 1:657, 1992.

Ron, et al., Mol. Cell. Biol, 2887-2895, 1991.

Roux et al., Proc. Nat'l Acad. Sci. USA, 86:9079-9083, 1989.

Rozen & Baratz, Cancer 49:1500-1503, 1982.

Rustgi, New England Journal of Medicine 331, 1694-1702, 1994.

Sachatello et al., Gastroenterology 58:699-708, 1970.

Sambrook et al., In: Molecular Cloning: A Laboratory Manual, 2d Ed., Cold Spring Harbor Laboratory Press, Cold Spring Harbor, N.Y., 1989.

Samulski et al., EMBO J., 10:3941-3950,1991.

Samulski et al., J. Virol., 61(10):3096-3101, 1987.

Schutte et al., Cancer Res., 56:2527, 1996.

Scott-Conner et al., Journal of the American College of Surgeons 181, 407-413, 1995.

Sekelsky et al., Genetics, 139:1347, 1995.

Shi et al., Nature, 388:87, 1997.

Shoemaker et al., Nature Genetics 14:450-456, 1996.

Silverman et al., Cytogenetics and Cell Genetics 75, 111-131, 1996.

Sirard et al. Targeted disruption in murine cells reveals variable requirement for Smad4 in transforming growth factor beta-related signaling. J. Biol. Chem. 275, 2063-2070, 2000.

Speigelman, et al., J. Biol. Chem., 264(3), 1811-1815, 1989.

Stemper et al., Ann Intern Med 83:639-646, 1975.

Takagi et al., Gastroenterology, 111: 1369, 1996.

Takaku et al., Cell, 92:645, 1998.

Temin, In: Gene Transfer, Kucherlapati (ed.), New York: Plenum Press, pp. 149-188, 1986.

ten Dijke et al., Activin receptor-like kinases: a novel subclass of cell-surface receptors with predicted serine/threonine kinase activity. Oncogene 8, 2879-2887, 1993.

Thiagalingam et al., Nature Genetics 13, 343-346,1996.

Thiagalingam et al., Mechanisms underlying losses of heterozygosity in human colorectal cancers. Proc. Nat'l Acad. Sci. USA 98, 2698-2702, 2001.

Thomas et al., Am J Hum Genet 58:770-776, 1996.

Tibbetts, Cell, 12:243-249, 1977.

Tur-Kaspa et al., Mol. Cell Biol., 6:716-718, 1986.

U.S. Pat. No. 3,817,837.

U.S. Pat. No. 3,850,752.

U.S. Pat. No. 3,939,350.

U.S. Pat. No. 3,996,345.

U.S. Pat. No. 4,275,149.

U.S. Pat. No. 4,277,437.

U.S. Pat. No. 4,340,535.

U.S. Pat. No. 4,366,241.

U.S. Pat. No. 4,683,195.

U.S. Pat. No. 4,683,202.

U.S. Pat. No. 4,786,600.

U.S. Pat. No. 4,800,159.

U.S. Pat. No. 4,873,191.

U.S. Pat. No. 4,883,750.

U.S. Pat. No. 4,988,617.

U.S. Pat. No. 5,712,097.

U.S. Pat. No. 5,712,097.

U.S. Pat. No. 5,712,097.

U.S. Pat. No. 5,190,856.

U.S. Pat. No. 5,270,184.

U.S. Pat. No. 5,279,721.

U.S. Pat. No. 5,324,631.

U.S. Pat. No. 5,359,046.

U.S. Pat. No. 5,484,699.

U.S. Pat. No. 5,494,810.

U.S. Pat. No. 5,496,699.

U.S. Pat. No. 5,633,365.

U.S. Pat. No. 5,639,611.

U.S. Pat. No. 5,665,549.

U.S. Pat. No. 5,712,124.

U.S. Pat. No. 5,733,733.

U.S. Pat. No. 5,733,752.

U.S. Pat. No. 5,744,311.

U.S. Pat. No. 5,747,255.

U.S. Pat. No. 5,747,469.

U.S. Pat. No. 6,207,814.

Umbas et al., Cancer Res., 52:5104-5109, 1992.

Veale et al., J. Med. Genet. 3:5-16, 1966.

Vogelstein et al., New England Journal of Medicine 319, 525-532, 1988.

Vogelstein, et al., Genes Chromosomes Cancer, 2:2, 159-162, 1990.

Wagner et al., Proc. Nat'l Acad. Sci. 87, 9:3410-3414, 1990.

Wagner et al., Science, 260:1510-1513,1993.

Walker et al., Proc. Nat'l Acad. Sci. USA, 89:392-396 1992.

Walpole et al., Am. J. Med. Genet. 32:1-8, 1989.

Walter et al., Nature Genetics 7, 22-28, 1994.

Walther & Stein, J. Mol. Med., 74:379-392, 1996.

Watanabe et al., Gastroenterology 77:148-151, 1979.

Watt et al., Proc. Nat'l Acad. Sci., 83(2): 3166-3170, 1986.

Whitelaw et al., Gastroenterology 112, 327-334, 1997.

Whitman, Gene Dev., 12, 2445-2462, 1998.

Wilson et al., Mol. Cell. Biol., 6181-6191, 1990.

WO 84/03564.

WO 90/07641

Wong et al., Gene, 10:87-94, 1980.

Woodford-Richens et al., Allelic loss at SMAD4 in polyps from juvenile polyposis patients and use of fluorescence in situ hybridization to demonstrate clonal origin of the epithelium. Cancer Res. 60, 2477-2482, 2000.

Wrana & Attisano, Trends Genet., 12:493, 1996.

Wrana & Pawson, Nature, 388:28, 1997.

Wu & Wu, Adv. Drug Delivery Rev., 12:159-167, 1993.

Wu & Wu, J. Biol. Chem., 262:4429-4432, 1987.

Wu & Wu, Biochem., 27:887-892, 1988.

Wu et al., Genomics, 4:560, 1989.

Yang et al., Proc. Nat'l Acad. Sci. USA, 87:9568-9572, 1990.

Yoshida et al., Endoscopy 20, 33-35, 1988.

Zechner et al., Mol. Cell. Biol., 2394-2401, 1988.

What is claimed is:

1. A method of diagnosing juvenile polyposis comprising the steps of:
(i) obtaining a sample from a subject; and
(ii) determining the loss or alteration of a functional BMPR1A gene in cells of said sample.

2. The method of claim 1 said sample is a tissue or fluid sample.

3. The method of claim 2, wherein said fluid is blood, buccal smear, or amniotic fluid.

4. The method of claim 1, wherein said determining comprises assaying for a nucleic acid from said sample.

5. The method of claim 4, further comprising subjecting said sample to conditions suitable to amplify said nucleic acid.

6. The method of claim 1, wherein said determining comprises contacting said sample with an antibody that binds immunologically to a BMPR1A.

7. The method of claim 6, further comprising subjecting proteins of said sample to ELISA.

8. The method of claim 1, further comprising the step of comparing the expression of BMPR1A in said sample with the expression of BMPR1A in non-juvenile polyposis samples.

9. The method of claim 8, wherein the comparison involves evaluating the level of BMPR1A expression.

10. The method of claim 8, wherein the comparison involves evaluating the structure of the BMPR1A gene, protein or transcript.

11. The method of claim 10, wherein said evaluating is an assay selected from the group consisting of sequencing, wild-type oligonucleotide hybridization, mutant oligonucleotide hybridization, SSCP, PCR™, denaturing gradient gel electrophoresis and RNase protection.

12. The method of claim 11, wherein said evaluating is wild-type or mutant oligonucleotide hybridization and said oligonucleotide is configured in an array on a chip or wafer.

13. The method of claim 1, wherein said juvenile polyposis sample comprises a mutation in the coding sequence of BMPR1A.

14. The method of claim 13, wherein said mutation produces a deletion mutant, an insertion mutant, a frameshift mutant, a nonsense mutant, a missense mutant or splice mutant.

15. The method of claim 13, wherein said mutation is a frameshift mutation.

16. The method of claim 15, wherein said mutation results in a premature termination of the BMPR1A gene product.

17. The method of claim 14, wherein said mutation is a missense mutation.

18. The method of claim 17, wherein said missense mutation results in an amino acid change of C82Y.

19. The method of claim 17, wherein said missense mutation results in an amino acid change of Q239X.

20. The method of claim 17, wherein said missense mutation results in an amino acid change of W271X.

21. The method of claim 17, wherein said missense mutation results in an amino acid change of Q17X.

22. The method of claim 17, wherein said missense mutation results in an amino acid change of E84X.

23. The method of claim 17, wherein said missense mutation results in an amino acid change of Y62D.

24. The method of claim 17, wherein said missense mutation results in an amino acid change of A338D.

25. The method of claim 18, wherein said missense mutation is 184T→G.

26. The method of claim 19, wherein said missense mutation is 715C→T.

27. The method of claim 20, wherein said missense mutation is 812G→A.

28. The method of claim 21, wherein said missense mutation is 349C→T.

29. The method of claim 22, wherein said missense mutation is 262G→T.

30. The method of claim 23, wherein said missense mutation is 184T→G.

31. The method of claim 24, wherein said missense mutation is 1013 C→A.

32. The method of claim 16, wherein said premature termination is at residue 122-123.

33. The method of claim 16, wherein said premature termination is at residue 363-364.

34. The method of claim 16, wherein said premature termination is at residue 35-36.

35. The method of claim 16, wherein said premature termination is at residue 321-322.

36. The method of claim 16, wherein said premature termination is at residue 259-260.

37. The method of claim 32, wherein said mutation is 353delT.

38. The method of claim 33, wherein said mutation is 1061delG.

39. The method of claim 34, wherein said mutation is 44-47delTGTT.

40. The method of claim 35, wherein said mutation is 961delC.

41. The method of claim 36, wherein said mutation is 674delT.

42. The method of claim 14, wherein said mutation is a splice mutation.

43. The method of claim 42, wherein said splice mutation results in a loss of exon 7 splice site.

44. The method of claim 43, wherein said splice mutation is 864-868delACTTG and IVS7+1−2delGT.

45. The method of claim 1, wherein said subject is characterized by one or more of:
(a) five juvenile polyps of the colorectum;
(b) junvile polyps throughout the gastrointestinal tract; and
(c) a family history of juvenile polyposis.

46. The method of claim 45, wherein said subject is characterized by two of (a)-(c).

47. The method of claim 45, wherein said subject is characterized by each of (a)-(c).

48. A method for altering the phenotype of a cell in a subject having juvenile polyposis comprising the step of contacting said cell with a functional BMPR1A under conditions permitting the uptake of said BMPR1A by said cell.

49. The method of claim 48, wherein said cell is derived from a gastrointestinal cell.

50. The method of claim 48, wherein said phenotype is selected from the group consisting of proliferation, migration, contact inhibition, soft agar growth and cell cycling.

51. The method of claim 48, wherein said BMPR1A is encapsulated in a liposome.

52. A method for altering the phenotype of a cell in a subject having juvenile polyposis comprising the step of contacting the cell with a nucleic acid (i) encoding BMPR1A and (ii) a promoter active in said cell, wherein said promoter is operably linked to the region encoding said BMPR1A, under conditions permitting the uptake of said nucleic acid by said cell.

53. The method of claim 52, wherein said cell is derived from a gastrointestinal cell.

54. The method of claim 52, wherein said cell is a tumor cell.

55. The method of claim 52, wherein the a phenotype is selected from the group consisting of proliferation, migration, contact inhibition, soft agar growth or cell cycling.

56. The method of claim 52, wherein said nucleic acid is encapsulated in a liposome.

57. The method of claim 56, wherein said nucleic acid is a viral vector selected from the group consisting of retrovirus, adenovirus, adeno-associated virus, vaccinia virus and herpesvirus.

58. The method of claim 57, wherein said nucleic acid is encapsulated in a viral particle.

59. A method for treating juvenile polyposis comprising the step of contacting a cell within a subject with BMPR1A under conditions permitting the uptake of said BMPR1A by said cell.

60. The method of claim 59, wherein the subject is a human.

61. A method for treating juvenile polyposis in a subject comprising the step of contacting a cell within said subject with a nucleic acid (i) encoding BMPR1A and (ii) a promoter active in said cell, wherein said promoter is operably linked to the region encoding said BMPR1A, under conditions permitting the uptake of said nucleic acid by said cell.

62. The method of claim 61, wherein said cell is derived from a tissue selected from the group consisting of skin, muscle, fascia, brain, prostate, breast, endometrium, lung, head & neck, pancreas, small intestine, blood cells, liver, testes, ovaries, colon, rectum, skin, stomach, esophagus, spleen, lymph nodes, bone marrow and kidney.

63. A method of diagnosing colorectal carcinoma comprising the steps of:
(i) obtaining a sample from a subject; and
(ii) determining the loss or alteration of a functional BMPR1A gene in cells of said sample.

64. The method of claim 63, wherein said sample is selected from the group consisting of blood, buccal smear, amniocentesis sample.

65. The method of claim 63, said sample comrising a tissue or fluid sample.

66. The method of claim 63, wherein said determining comprises assaying for a BMPR1A nucleic acid from said sample.

67. The method of claim 66, further comprising subjecting said sample to conditions suitable to amplify said nucleic acid.

68. The method of claim 63, wherein said determining comprises contacting said sample with an antibody that binds immunologically to a BMPR1A.

69. The method of claim 68, further comprising subjecting proteins of said sample to ELISA.

70. The method of claim 63, further comprising the step of comparing the expression of BMPR1A in said sample with the expression of BMPR1A in non-colorectal cancer samples.

71. The method of claim 70, wherein the comparison involves evaluating the level of BMPR1A expression.

72. The method of claim 70, wherein the comparison involves evaluating the structure of the BMPR1A gene, protein or transcript.

73. The method of claim 72, wherein said evaluating is an assay selected from the group consisting of sequencing, wild-type oligonucleotide hybridization, mutant oligonucleotide hybridization, SSCP, PCR™, denaturing gradient gel electrophoresis, antibody binding and RNase protection.

74. The method of claim 72, wherein said evaluating is wild-type or mutant oligonucleotide hybridization and said oligonucleotide is configured in an array on a chip or wafer.

75. The method of claim 63, wherein said colorectal carcinoma sample comprises a mutation in the coding sequence of BMPR1A.

76. The method of claim 63, wherein said loss or alteration of function is caused by a deletion mutant, an insertion mutant, a frameshift mutant, a nonsense mutant, a missense mutant or splice mutant.


SEQUENCE LISTING (2 sequences)

SEQ ID NO: 1 - DNA, 2623 bp, Artificial Sequence
  CDS (1)..(1596): Description of Artificial Sequence  Synthetic Primer
atgactcagctatacatttacatcagattattgggagcctatttgttcatcatttctcgtgttcaaggacagaatctggatagtatgcttcatggcactgggatgaaatcagactccgaccagaaaaagtcagaaaatggagtaaccttagcaccagaggataccttgccttttttaaagtgctattgctcagggcactgtccagatgatgctattaataacacatgcataactaatggacattgctttgccatcatagaagaagatgaccagggagaaaccacattagcttcagggtgtatgaaatatgaaggatctgattttcagtgcaaagattctccaaaagcccagctacgccggacaatagaatgttgtcggaccaatttatgtaaccagtatttgcaacccacactgccccctgttgtcataggtccgttttttgatggcagcattcgatggctggttttgctcatttctatggctgtctgcataattgctatgatcatcttctccagctgcttttgttacaaacattattgcaagagcatctcaagcagacgtcgttacaatcgtgatttggaacaggatgaagcatttattccagttggagaatcactaaaagaccttattgaccagtcacaaagttctggtagtgggtctggactacctttattggttcagcgaactattgccaaacagattcagatggtccggcaagttggtaaaggccgatatggagaagtatggatgggcaaatggcgtggcgaaaaagtggcggtgaaagtattctttaccactgaagaagccagctggtttcgagaaacagaaatctaccaaactgtgctaatgcgccatgaaaacatacttggtttcatagcggcagacattaaaggtacaggttcctggactcagctctatttgattactgattaccatgaaaatggatctctctatgacttcctgaaatgtgctacactggacaccagagccctgcttaaattggcttattcagctgcctgtggtctgtgccacctgcacacagaaatttatggcacccaaggaaagcccgcaattgctcatcgagacctaaagagcaaaaacatcctcatcaagaaaaatgggagttgctgcattgctgacctgggccttgctgttaaattcaacagtgacacaaatgaagttgatgtgcccttgaataccagggtgggcaccaaacgctacatggctcccgaagtgctggacgaaagcctgaacaaaaaccacttccagccctacatcatggctgacatctacagcttcggcctaatcatttgggagatggctcgtcgttgtatcacaggagggatcgtggaagaataccaattgccatattacaacatggtaccgagtgatccgtcatacgaagatatgcgtgaggttgtgtgtgtcaaacgtttgcggccaattgtgtctaatcggtggaacagtgatgaatgtctacgagcagttttgaagctaatgtcagaatgctgggcccacaatccagcctccagactcacagcattgagaattaagaagacgcttgccaagatggttgaatcccaagatgtaaaaatctgatggttaaaccatcggaggagaaactctagactgcaagaactgtttttacccatggcatgggtggaattagagtggaataaggatgttaacttggttctcagactctttcttcactacgtgttcacaggctgctaatattaaacctttcagtactcttattaggatacaagctgggaacttctaaacacttcattctttatatatggacagctttattttaaatgtggtttttgatgcctttttttaagtgggtttttatgaactgcatcaagacttcaatcctgattagtgtctccagtcaagctctgggtactgaattgcctgttcataaaacggtgctttctgtgaaagccttaagaagataaatgagcgcagcagagatggagaaatagactttgccttttacctgagacattcagttcgtttgtattctacctttgtaaaacagcctatagatgatgatgtgtttgggatactgcttattttatgatagtttgtcctgtgtccttagtgatgtgtgtgtgtctccatgcacatgcacgccgggattcctctgctgccatttgaattagaagaaaataatttatatgcatgcacaggaagatattggtggccggtggttttgtgctttaaaaatgcaatatctgaccaagattcgccaatctcatacaagccatttactttgcaagtgagatagcttccccaccagctttattttttaacatgaaagctgatgccaaggccaaaagaagtttaaagcatctgtaaatttggactgttttccttcaaccaccattttttttgtggttattatttttgtcacggaaagcatcctctccaaagttggagcttctattgccatgaaccatgcttacaaagaaagcacttcttattgaagtgaattcctgcatttgatagcaatgtaagtgcctataaccatgttctatattctttattctcagtaacttttaaaagggaagttatttatattttgtgtataatgtgctttatttgcaaatcaccc

SEQ ID NO: 2 - protein, 532 aa, Artificial Sequence
  Description of Artificial Sequence  Synthetic
MTQLYIYIRLLGAYLFIISRVQGQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRWLVLLISMAVCIIAMIIFSSCFCYKHYCKSISSRRRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDVPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKI